(19) United States
(12) Patent Application Publication
(10) Pub. No.: US 2015/0329860 A1
(43) Pub. Date: Nov. 19, 2015

(54) GENE SILENCING

(71) Applicant: THE ROCKEFELLER UNIVERSITY, New York, NY (US)

(72) Inventors: Nam-Hai CHUA, New York, NY (US); Qiwen NIU, Staten Island, NY (US); Shulin DENG, New York, NY (US)

(73) Assignee: THE ROCKEFELLER UNIVERSITY, New York, NY (US)

(21) Appl. No.: 14/652,518

(22) PCT Filed: Dec. 17, 2013
(86) PCT No.: PCT/US2013/075720

§ 371 Date: Jun. 16, 2015

Related U.S. Application Data
(60) Provisional application No. 61/738,651, filed on Dec. 18, 2012.

(51) Int. Cl. C12N 15/113; A01G 1/00; C12N 15/82

(52) U.S. Cl. CPC C12N 15/113; C12N 15/8218; A01G 1/001

(57) ABSTRACT

The present invention relates to transcriptional gene silencing (TGS) of endogenes in plants, plant tissue and plant cells. More specifically, the present invention relates to nucleic acid constructs that are capable of more effectively silencing genes of interest, such as endogenes, in plants, plant tissue and plant cells by TGS. The present invention further relates to methods of more effectively reducing endogenous gene expression in plants, plant tissues or plant cells by TGS using the nucleic acid constructs of the invention.

### CROSS-REFERENCE TO RELATED APPLICATION

The present application claims priority to U.S. provisional patent application Ser. No. 61/738,651 file on 18 Dec. 2012. This application is incorporated herein by reference.

### SEQUENCE SUBMISSION

The present application is being filed along with a Sequence Listing in electronic format. The Sequence Listing is entitled 2312128PCTSequenceListing.txt, created on 5 Dec. 2013 and is 27 kb in size. The information in the electronic format of the Sequence Listing is incorporated herein by reference in its entirety.

### BACKGROUND OF THE INVENTION

The present invention relates to transcriptional gene silencing (TGS) of endogenes in plants, plant tissue and plant cells. More specifically, the present invention relates to nucleic acid constructs that are capable of more effectively silencing genes of interest, such as endogenes, in plants, plant tissue and plant cells by TGS. The present invention further relates to methods of more effectively reducing endogenous gene expression in plants, plant tissues or plant cells by TGS using the nucleic acid constructs of the invention.

The publications and other materials used herein to illuminate the background of the invention or provide additional details respecting the practice, are incorporated by reference, and for convenience are respectively grouped in the Bibliography.

More than a decade ago, Matzke and colleagues reported the relationship between transcriptional gene silencing (TGS) of a transgene promoter (NOS) in plants and methylation on the promoter DNA (Matzke et al., 1989). Since then, mechanisms underlying TGS in plants have been extensively studied for both transgenes and endogenes in monocots and dicots (for reviews, see (Matzke and Matzke, 2004; Eamens et al., 2008; Matzke et al., 2009)). The current view of TGS suggests that 21-24 nt small RNAs (sRNAs) generated by double-stranded RNA processing machinery (AGO4, DCL3) are targeted to genomic regions with sequence homology. These sRNAs guide DNA methylation by the action of DNA methyltransferases (DRM1/2, MET1, CMT3) and presumably this is followed by histone modifications involving histone methyltransferase and deacetylase so as to establish a repressive chromatin state at the methylated locus.

Early studies using transgenes as a model system uncovered some basic features related to TGS in plants (Mette et al., 2000; Jones et al., 2001; Sijen et al., 2001). These studies demonstrated that long hairpin structures are required to generate sRNAs targeted to transgene promoters. DNA analysis of the silenced transgenes showed increases in both symmetrical and asymmetrical cytosine methylations and the involvement of MET1 in the maintenance of the silenced locus. Similarly, long inverted repeats (IR) have also been shown to mediate efficient TGS of a 35S promoter in rice (Okano et al., 2008). Notably, single-stranded silencers in the sense (S) or antisense (AS) orientation were inefficient in producing sRNAs and generating TGS in tobacco and Arabidopsis (Mette et al., 2000).

Although the sequence of events surrounding TGS of transgenes is relatively well defined it is not known whether similar steps also apply to TGS of endogenous loci (endogenes hereinafter). Surprisingly, only a few cases of TGS of endogenous loci have been reported to-date, all of which used IR RNA's. Cigan et al. (2005) have reported successful and strong silencing of two maize endogenous genes (Cigan et al., 2005). Moderate silencing was seen for a few endogenes in petunia, potato and rice (Sijen et al., 2001; Heilersig et al., 2006; Okano et al., 2008). The inefficiency of IR RNA's to trigger TGS of endogenes was well illustrated in a comprehensive study using the model monocot rice. Interestingly, 6 out of 7 endogenes examined in rice appeared to be recalcitrant to silencing by sRNAs generated from IR RNA structures (Okano et al., 2008). By contrast, in the same experiment it was shown that a 35S promoter could be efficiently silenced by IR RNA targeted to this promoter. Other unsuccessful trials (phytoene desaturase, (PDS) and Chalcone synthase (CHS)) have also been reported in Arabidopsis (Eamens et al., 2008). Together, these results suggest endogenous loci may possess some intrinsic properties that can prevent unexpected TGS; alternatively, there is the need to explore more efficient silencing strategies for endogenous loci. Other than the observed low success rate of gene silencing at endogenous loci, there is also a discrepancy between TGS and DNA methylation as well as between DNA methylation and histone modifications for endogenes (Okano et al., 2008). For most of the cases in rice, IR RNA's targeted to promoter regions could trigger DNA methylation of homologous sequences but they failed to induce chromatin modifications and TGS (Okano et al., 2008).

It is desired to develop nucleic acid constructs and methods of enhancing transcriptional gene silencing in plants.

### SUMMARY OF THE INVENTION

The present invention relates to transcriptional gene silencing (TGS) of endogenes in plants, plant tissue and plant cells. More specifically, the present invention relates to nucleic acid constructs that are capable of more effectively silencing genes of interest, such as endogenes, in plants, plant tissue and plant cells by TGS. The present invention further relates to methods of more effectively reducing endogenous gene expression in plants, plant tissues or plant cells by TGS using the nucleic acid constructs of the invention.

In a first aspect, the present invention provides a nucleic acid construct comprising a plant operable promoter as described herein operably linked to a nucleic acid molecule that comprises a silencing enhancer described herein operatively linked to a nucleic acid silencer molecule described herein. The nucleic acid construct may optionally include other regulatory sequences, such as 3′ regulatory sequences, or other sequences as described herein. In accordance with this aspect, the present invention also provides an isolated silencing enhancer as described herein and a nucleic acid construct comprising a plant operable promoter as described herein operably linked to a nucleic acid molecule that comprises a silencing enhancer described herein. In some embodiments, the silencing enhancer is a region of the promoter of SUPPRESSOR OF ddc (SDC). In one embodiment, the silencing enhancer comprises the sequence set forth in SEQ ID NO:6. In another embodiment, the silencing enhancer comprises the sequence set forth in SEQ ID NO:3. In a further embodiment, the silencing enhancer comprises nucleotides 1-389 of the sequence set forth in SEQ ID NO:1 and any number of nucleotides that are contiguous and are 3′ and contiguous to nucleotide 389. In some embodiments, the silencing enhancer comprises the sequence set forth in SEQ ID NO:1, SEQ ID NO:4, SEQ ID NO:5 or SEQ ID NO:18. In other embodiments, the silencing enhancer consists of the sequence set forth in SEQ ID NO:1, SEQ ID NO:3, SEQ ID NO:4, SEQ ID NO:5, SEQ ID NO:6 or SEQ ID NO:18.

In some embodiments, the silencing enhancer is a region of the promoter of SUPPRESSOR OF ddc (SDC). In another embodiment, the silencing enhancer may comprise a nucleic acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO:1, 3, 4, 5, 6 or 18. The silencing enhancer may comprise a nucleotide sequence that is hybridizable under stringent conditions with a DNA molecule comprising the full complement of SEQ ID NO:1, 3, 4, 5, 6 or 18. The silencing enhancer may comprise a nucleotide sequence, wherein the nucleotide sequence is derived from SEQ ID NO:1, 3, 4, 5, 6 or 18 by alteration of one or more nucleotides by at least one method selected from the group consisting of: deletion, substitution, addition and insertion. The silencing enhancer may comprise a nucleotide sequence, wherein the nucleotide sequence corresponds to an allele of the promoter of SUPPRESSOR OF ddc (SDC).

In one embodiment, the nucleic acid silencer molecule of the present invention comprises a promoter region of a plant endogene target, i.e., a plant endogene to be downregulated via TGS. The nucleic acid silencer molecule of this first embodiment encodes either a single-stranded silencer which is an RNA molecule transcribed from the nucleic acid construct or an inverted repeat silencer transcribed from the nucleic acid construct. The single-stranded silencer or inverted repeat silencer provides TGS of endogenes in plants, plant tissues and plant cells. In one embodiment, the single-stranded silencer is an RNA molecule that is produced from a promoter region of a plant endogene target (i.e., a nucleic acid silencer molecule) that is in an antisense orientation with respect to the plant operable promoter in the nucleic acid construct. In another embodiment, the single-stranded silencer is an RNA molecule that is produced from a promoter region of a plant endogene target (i.e., a nucleic acid silencer molecule) that is in a sense orientation with respect to the plant operable promoter in the nucleic acid construct. In each of these embodiments, the construct, nucleic acid silencer molecule and single-stranded silencers are in the absence of inverted repeat structures, i.e., no inverted repeat structures or inverted repeats are present in the nucleic acid construct and products produced from it. In yet another embodiment, the nucleic acid silencer is an RNA molecule that is produced from a promoter region of a plant endogene target that is provided in duplicate and arranged in an inverted repeat configuration with respect to the plant operable promoter in the nucleic acid construct. Expression of the nucleic acid silencer molecule produces an initial single stranded RNA. This single stranded RNA may be converted to a double stranded RNA by cellular mechanisms or as a result of the inverted repeat structure.

In one embodiment, sRNAs are produced from the expressed nucleic acid silencer molecule in a cell containing the nucleic acid silencer molecule. In another embodiment, the silencing enhancer enhances the production of sRNAs from the expressed nucleic acid silencer molecule in a cell containing the silencing enhancer operatively linked to the nucleic acid silencer molecule.

In one embodiment, the nucleic acid silencer molecule comprises nucleotides upstream of the transcription start site of the target endogene. In another embodiment, the nucleic acid silencer molecule comprises nucleotides upstream of the transcription start site and nucleotides downstream of the transcription start site of the target endogene. In some embodiments, the nucleic acid silencer molecule comprises a promoter region of about 300 contiguous nucleotides to about 1500 contiguous nucleotides of the endogene. In other embodiments, the nucleic acid silencer molecule comprises a promoter region of about 400 contiguous nucleotides to about contiguous 1200 nucleotides of the endogene. In additional embodiments, the nucleic acid silencer molecule comprises a promoter region of about 425 contiguous nucleotides to about 1100 contiguous nucleotides of the endogene. In further embodiments, the nucleic acid silencer molecule comprises a promoter region of about 425 contiguous nucleotides to about 1075 contiguous nucleotides of the endogene.

In one embodiment, the nucleic acid silencer molecule of the present invention comprises a promoter region of a plant endogene target, i.e., a plant endogene to be downregulated via TGS. In another embodiment, the nucleic acid silencer molecule comprise the 2000 nucleotides immediately 5′ to the transcription start site of the endogene to be downregulated, or a fragment of this 2000 nucleotide region. The fragment may comprise at least 100, 200, 300, 400, 500, 600, 700, 800, 900, 1000, 1100, 1200, 1300, 1400, 1500, 1600, 1700, 1800, 1900 or 2000 contiguous nucleotides of the region that is 2000 nucleotides immediately 5′ to the transcription start site of the endogene to be downregulated.

Any promoter that is operable in a plant may be used in the nucleic acid construct to drive expression of the nucleic acid molecule. In some embodiments, the promoter is a single copy of a plant operable promoter, including those described herein. In other embodiments, the promoter is a double copy of a plant operable promoter to make a homologous double promoter. In further embodiments, the promoter is a combination of two different promoters to make a heterologous double promoter.

In a second aspect, the present invention provides a transgenic plant cell comprising the nucleic acid construct. In one embodiment, the nucleic acid construct is stably integrated into the genome of the transgenic plant cell. In another embodiment, the nucleic acid is expressed in the transgenic plant cell.

In a third aspect, the present invention provides a transgenic plant comprising the nucleic acid construct. In one embodiment, the nucleic acid construct is stably integrated into the genome of the transgenic plant. In another embodiment, the nucleic acid is expressed in the transgenic plant.

In a fourth aspect, the present invention provides a method of more effectively silencing a gene of interest, such as more effectively silencing endogenous gene expression, in plants, plant tissues or plant cells through transcriptional gene silencing. In one embodiment, the method comprises transfecting a plant cell with a nucleic acid construct to produce a transgenic plant cell as described herein. The method further comprises expressing the nucleic acid silencer molecule described herein in the transgenic plant cell as described herein. The expressed nucleic acid silencer molecule described herein is cleaved in the transgenic plant cell to produce one or more small RNAs (sRNAs) that induces transcriptional gene silencing to reduce expression of the target gene of interest. In some embodiments, expression of the nucleic acid silencer molecule produces an initial single stranded RNA. This single stranded RNA may be converted to a double stranded RNA before processing to produce sRNAs by cellular mechanisms or as a result of the inverted repeat structure. The method may optionally include preparing a nucleic acid construct encoding a nucleic acid as described herein. In another embodiment, the method comprises regenerating a transgenic plant from the transgenic plant cell. In this embodiment, the nucleic acid silencer molecule is expressed in the transgenic plant. The expressed nucleic acid silencer molecule is cleaved in the transgenic plant cell to produce one or more sRNAs that induces transcriptional gene silencing to reduce expression of the target gene of interest.

In a fifth aspect, the present invention provides nucleic acid constructs and methods to identify and obtain other silencing enhancers for use in plant TGS. According to this aspect, the nucleic acid construct is one that is suitable for transformation of a plant species for which it is desired to identify a silencing enhancer. In one embodiment, the nucleic acid construct may be included in a vector. In some embodiments, the vector is suitable for Agrobacterium-mediated transformation. In other embodiments, the vector may be suitable for biolistic-mediated transformation. Other suitable vectors for plant transformations are well known to the skilled artisan. In another embodiment, the nucleic acid construct may be used directly for the transformation of a plant according to techniques well known to the skilled artisan. The nucleic acid construct comprises a plant operable promoter operatively linked to a putative silencing enhancer operatively linked to a nucleic acid silencer molecule described herein operatively linked to plant operable 3′ regulatory region.

In one embodiment, the polynucleotide to be tested for silencing enhancer activity is a region of the promoter of a homolog of SUPPRESSOR OF ddc (SDC) from a species other than Arabidopsis. The homolog of SDC may be from a monocot or a dicot. The homolog of SDC may be selected from the group consisting of: maize, soybean, sunflower, sorghum, canola, wheat, alfalfa, cotton, rice, barley, millet, sugar cane and switchgrass. In another embodiment, the putative silencing enhancer or polynucleotide which is to be tested for enhancing transcriptional gene silencing is a promoter region of a gene loci that is silenced by higher levels of endogenous 24 nt siRNAs and is heavily methylated. In one embodiment, the plant operable promoter is a single promoter, a double homologous promoter or a double heterologous promoter. In one embodiment, the plant operable promoter is a single or a double 35S CMV promoter. In one embodiment, the 3′ sequence is a TRV2 3′ sequence. In an additional embodiment, the 3′ regulatory region is a polyA addition sequence. In one embodiment, the polyA sequence is a NOS poly A sequence. The testing for more effectively silencing of a target gene may be carried out according to methods well known in the art. These methods include, but are not limited to, RT-PCR, PCR, northern blot analysis, immunological assay and enzymatic assay. The testing of fragments of the promoter region to be tested for more effective silencing may be carried out according to methods described herein.

### BRIEF DESCRIPTION OF THE FIGURES

FIG. 1 shows schematic representations of different truncated SDC promoters.

FIGS. 2a-2d show the stomata cluster phenotype. FIG. 2a: Stomata pattern in wild type (WT). FIG. 2b: Stomata clusters in pMA/PTMM plants. FIGS. 2c and 2d: Stomata clusters in pBA/PSDC-PTMM plants.

FIG. 3 shows qPCR of TMM RNA levels. Numbers refer to different transformed lines and lines with clustered stomata phenotype are marked with asterisks.

FIGS. 4a-4c show DNA methylation profiles at the endogenous TMM promoter. FIG. 4a: The region of endogenous TMM promoter analyzed by sequencing of bisulfite-treated DNA. FIGS. 4b and 4c: DNA methylation levels (%) at CG, CHG (H=A, C, or T), and CHH types of cytosine, respectively in different transformed lines.

FIGS. 5a-5f show histone H3 modification patterns on the TMM promoter and coding region in pBA/PSDC(-DR)-PTMM plants. FIG. 5a: qPCR primers designed to amplify DNA fragments (around 100 bp) corresponding TMM promoter and coding sequences as indicated. FIGS. 5b-5f: Different H3 modification in TMM promoter and coding region. Three lines of pBA/PSDC(-DR)-PTMM plants were tested. Clustered stomata phenotypes were observed in both line #4 and 9 but not in line #12.

FIGS. 6a-6c show Northern blots for PTMM-related siRNAs. FIG. 6a: Northern blot for tobacco N. benthemiana transformation. FIG. 6b: Northern blot for Arabidopsis stable transformed lines of pBA/PTMM. FIG. 6c: Northern blot for Arabidopsis stable transformed lines of pBA/PSDC(-DR)-PTMM. In FIGS. 6c and 6b, sample c is positive control.

FIGS. 7a-7c show small RNAs analysis in different TGS plants. FIG. 7a: Cloned numbers and size distribution of smRNAs mapped to the TMM promoter region. FIG. 7b: Small RNAs distribution in plus and minus strands of TMM promoter region. FIG. 7c: The first base distribution among different TGS plants.

FIGS. 8a-8d show small RNAs distribution in TMM promoter region. FIG. 8a: Small RNAs distribution on the TMM promoter region of pBA/PSDC(-DR)-PTMM plants. FIG. 8b: Small RNAs distribution on the TMM promoter region of pBA/PTMM plants. FIG. 8c: Small RNAs distribution on the the the TMM promoter region of pBA/PTMM-AS plants. FIG. 8d: Small RNAs distribution on the TMM promoter region of pBA/PTMM-IR plants.

FIG. 9 shows epigenome file of SDC promoter. Epigenome file shows DNA methylation and small RNAs levels in WT (Col-0), met1, ddc, and rdd mutant. The first line (from up) shows the region of SDC (AT2G17690.1) ORF to its upstream neighbor ORF. PSDC sequence were divided into five regions named A, B, C, TR, and DR. Line 2 to 5 (from up) show three types (CG, CHG, and CHH) DNA methylation levels. In DR (direct repeats) region, a heavy DNA methylation is showed in WT and met, rdd mutants but not in ddc mutant. Small RNA levels are showed in Line 6 to 9 (from up). A abundance of small RNAs at DR region in WT but abolished in ddc mutant. In A region, small RNAs increased in rdd mutant.

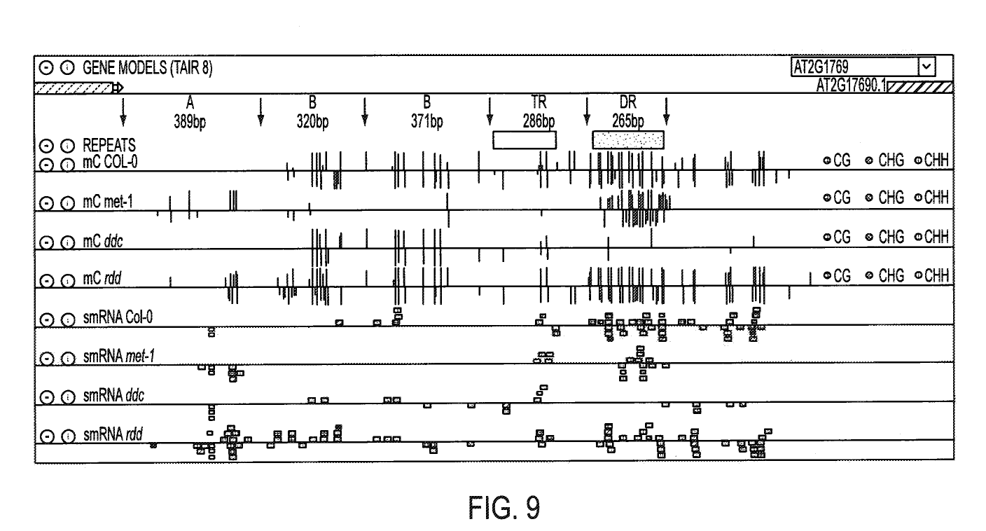

FIG. 10 shows Northern blot showing siRNAs levels promoted by different truncated SDC promoter sequences.

FIGS. 11a-11d show Northern blot to confirm region A enhances siRNA production related to other DNA fragments. FIG. 11a: Northern blot showing enhancement of PTMM-AS related siRNA production by region A. FIG. 11b: Northern blot showing enhancement of PCH42 related siRNA production by region A. FIGS. 11c and 11d: Northern result blot showing enhancement of PFAD2 related siRNA production by region A.

### DETAILED DESCRIPTION OF THE INVENTION

The present invention relates to transcriptional gene silencing (TGS) of endogenes in plants, plant tissue and plant cells. More specifically, the present invention relates to nucleic acid constructs that are capable of more effectively silencing genes of interest, such as endogenes, in plants, plant tissue and plant cells by TGS. The present invention further relates to methods of more effectively reducing endogenous gene expression in plants, plant tissues or plant cells by TGS using the nucleic acid constructs of the invention.

Unless defined otherwise, all technical and scientific terms used herein have the same meaning as is commonly understood by one of skill in the art to which the invention belongs.

The terms “polynucleotide,” “nucleotide sequence,” and “nucleic acid” are used to refer to a polymer of nucleotides (A, C, T, U, G, etc. or naturally occurring or artificial nucleotide analogues), e.g., DNA or RNA, or a representation thereof, e.g., a character string, etc., depending on the relevant context. A given polynucleotide or complementary polynucleotide can be determined from any specified nucleotide sequence.

A polynucleotide, polypeptide or other component is “isolated” when it is partially or completely separated from components with which it is normally associated (other proteins, nucleic acids, cells, synthetic reagents, etc.). A nucleic acid or polypeptide is “recombinant” when it is artificial or engineered, or derived from an artificial or engineered protein or nucleic acid. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

The term “nucleic acid construct” or “polynucleotide construct” means a nucleic acid molecule, either single- or double-stranded, which is isolated from a naturally occurring gene or which has been modified to contain segments of nucleic acids in a manner that would not otherwise exist in nature. The term nucleic acid construct is synonymous with the term “expression cassette” when the nucleic acid construct contains the control sequences required for expression of a sequence of the present invention.

The term “control sequences” is defined herein to include all components, which are necessary or advantageous for the expression of a polynucleotide of the present invention. Each control sequence may be native or foreign to the polynucleotide sequence. At a minimum, the control sequences include a promoter and transcriptional stop signals. The control sequences may be provided with linkers for the purpose of introducing specific restriction sites facilitating ligation of the control sequences to the nucleotide sequence.

The term “operably linked” is defined herein as a configuration in which a control sequence is appropriately placed at a position relative to the nucleotide sequence of the nucleic acid construct such that the control sequence directs the expression of a polynucleotide of the present invention.

In the present context, the term “expression” includes transcription of the polynucleotide. In the present context, the term “expression vector” covers a DNA molecule, linear or circular, that comprises a polynucleotide of the invention, and which is operably linked to additional segments that provide for its transcription.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

The term “heterologous” as used herein describes a relationship between two or more elements which indicates that the elements are not normally found in proximity to one another in nature. Thus, for example, a polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). An example of a heterologous polypeptide is a polypeptide expressed from a recombinant polynucleotide in a transgenic organism. Heterologous polynucleotides and polypeptides are forms of recombinant molecules.

The term “transfecting” as used herein refers to the deliberate introduction to a nucleic acid into a cell. Transfection includes any method known to the skilled artisan for introducing a nucleic acid into a cell, including, but not limited to, Agrobacterium infection, ballistics, electroporation, microinjection and the like.

The term “silencing enhancer” as used herein refers to a nucleic acid fragment that, when operatively linked to a nucleic acid silencer molecule, functions to provide more effective silencing of the target of the nucleic acid silencer molecule.

The term “nucleic acid silencer molecule” as used herein refers to a part of a nucleic acid construct in accordance with the present invention that comprises a promoter region of a target plant endogene. The nucleic acid silencer molecule is transcribed to initially produce a single-stranded RNA that is processed in the plant cell to produce small RNAs (sRNAs) that induce transcriptional gene silencing of the target plant endogene. The nucleic acid silencer molecule may be placed in a sense orientation or in an antisense orientation with respect to the plant operable promoter of the nucleic acid construct or it may be placed in an inverted repeat structure with respect to the plant operable promoter of the nucleic acid construct.

The term “single-stranded sense silencer” or “single-stranded S silencer” as used herein refers to a single stranded RNA produced by a nucleic acid silencer molecule in the sense orientation with respect to the promoter.

The term “single-stranded antisense silencer” or “single-stranded AS silencer” as used used herein refers to a single stranded RNA produced by a nucleic acid silencer molecule in the antisense orientation with respect to the promoter.

The term “inverted repeat silencer” or “IR silencer” as used herein refers to a RNA molecule produce by a nucleic acid silencer molecule having a two copies of the target endogene promoter sequence, one copy inverted with respect to the second copy and preferably separated by a spacer.

“Reduced gene expression” means that the expression of a plant endogene is reduced in a transgenic plant cell or transgenic plant containing a nucleic acid silencer molecule stably integrated in its genome when compared to a plant cell or plant which does not contain the nucleic acid silencer molecule. “Reduced gene expression” may involve a reduction of expression of a plant endogene by at least 10%, at least 20%, at least 30%, at least 40%, at least 50%, at least 60%, at least 70%, at least 75%, at least 80%, at least 85%, at least 90%, at least 95% or 100%.

“More effective gene silencing” as used herein means that the gene silencing of the target gene is more effective in the presence of the silencing enhancer than in its absence. More effective gene silencing may be measured by an increase in the reduction of gene expression. Alternatively, more effective gene silencing may be measured by an increase in the production of sRNAs from the nucleic acid silencer molecule. In addition, more effective gene silencing may be measured by an increase in penetration rates of the nucleic acid construct. Alternatively, more effective gene silencing may be measured by a reduction in off-target effects. The skilled artisan will also readily recognize other factors that can be measured to determine more effective gene silencing. The more effective gene silencing is seen with respect to the silencing caused by a nucleic acid silencer molecule in a plant or plant cell containing a nucleic acid construct comprising a silencing enhancer operatively linked to a nucleic acid silencer molecule that is stably integrated in its genome when compared to a plant cell or plant which contains only the nucleic acid silencer molecule stably integrated in its genome. In the context of an increase in the reduction of gene expression, reduced gene expression caused by a nucleic acid silencing molecule operatively linked to a silencing enhancer is greater by at least 5%, at least 10%, at least 20%, at least 30%, at least 40%, at least 50%, at least 60%, at least 70% or more. In the context of an increase in penetration rate caused by a nucleic acid silencing molecule operatively linked to a silencing enhancer is greater by at least 100%, at least 150%, at least 200%, at least 250%, at least 300%, at least 350%, at least 400%, at least 450%, at least 500%, at least 550%, at least 600%, or more.

Thus, in a first aspect, the present invention provides a nucleic acid construct comprising a plant operable promoter as described herein operably linked to a nucleic acid molecule that comprises a silencing enhancer described herein operatively linked to a nucleic acid silencer molecule described herein. The nucleic acid construct may optionally include other regulatory sequences, such as 3′ regulatory sequences, or other sequences as described herein. In accordance with this aspect, the present invention also provides an isolated silencing enhancer as described herein and a nucleic acid construct comprising a plant operable promoter as described herein operably linked to a nucleic acid molecule that comprises a silencing enhancer described herein. In some embodiments, the silencing enhancer is a region of the promoter of SUPPRESSOR OF ddc (SDC). In one embodiment, the silencing enhancer comprises the sequence set forth in SEQ ID NO:6. In another embodiment, the silencing enhancer comprises the sequence set forth in SEQ ID NO:3. In a further embodiment, the silencing enhancer comprises nucleotides 1-389 of the sequence set forth in SEQ ID NO:1 and any number of nucleotides that are contiguous and are 3′ and contiguous to nucleotide 389. In one embodiment, the silencing enhancer comprises nucleotides 1-390 of the sequence set forth in SEQ ID NO:1. In another embodiment, the synthetic promoter comprises nucleotides 1-600 of the sequence set forth in SEQ ID NO:1. In an additional embodiment, the silencing enhancer comprises nucleotides 1-709 of the sequence set forth in SEQ ID NO: 1. In a further embodiment, the silencing enhancer comprises nucleotides 1-810 of the sequence set forth in SEQ ID NO:1. In another embodiment, the silencing enhancer comprises nucleotides 1-1000 of the sequence set forth in SEQ ID NO:1. In an additional embodiment, the silencing enhancer comprises nucleotides 1-1120 of the sequence set forth in SEQ ID NO:1. In a further embodiment, the silencing enhancer comprises nucleotides 1-1225 of the sequence set forth in SEQ ID NO:1. In one embodiment, the silencing enhancer comprises nucleotides 1-1346 of the sequence set forth in SEQ ID NO:1. In another embodiment, the silencing enhancer comprises nucleotides 1-1466 of the sequence set forth in SEQ ID NO:1. In an additional embodiment, the silencing enhancer comprises nucleotides 1-1487 of the sequence set forth in SEQ ID NO: 1. In a further embodiment, the silencing enhancer comprises nucleotides 1-1592 of the sequence set forth in SEQ ID NO: 1. In another embodiment, the silencing enhancer comprises nucleotides 1-1663 of the sequence set forth in SEQ ID NO: 1. In a further embodiment, the silencing enhancer comprises nucleotides 1-1730 of the sequence set forth in SEQ ID NO: 1. In some embodiments, the silencing suppressor comprises the sequence set forth in SEQ ID NO:4, SEQ ID NO:5 or SEQ ID NO:18. These examples of silencing enhancers are exemplary only and illustrate that the inventors contemplate any silencing enhancer comprising 389-1731 contiguous nucleotides that include nucleotides 1-389 of SEQ ID NO:1. In some embodiments, the silencing enhancer comprises the sequence set forth in SEQ ID NO:1, SEQ ID NO:4, SEQ ID NO:5 or SEQ ID NO:18. In other embodiments, the silencing enhancer consists of the sequence set forth in SEQ ID NO:1, SEQ ID NO:3, SEQ ID NO:4, SEQ ID NO:5, SEQ ID NO:6 or SEQ ID NO:18.

In some embodiments, the silencing enhancer is a region of the promoter of SUPPRESSOR OF ddc (SDC). In another embodiment, the silencing enhancer may comprise a nucleic acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, when compared to SEQ ID NO:1, 3, 4, 5, 6 or 18. The silencing enhancer may comprise a nucleotide sequence that is hybridizable under stringent conditions with a DNA molecule comprising the full complement of SEQ ID NO:1, 3, 4, 5, 6 or 18. The silencing enhancer may comprise a nucleotide sequence, wherein the nucleotide sequence is derived from SEQ ID NO:1, 3, 4, 5, 6 or 18 by alteration of one or more nucleotides by at least one method selected from the group consisting of: deletion, substitution, addition and insertion. The silencing enhancer may comprise a nucleotide sequence, wherein the nucleotide sequence corresponds to an allele of the promoter of SUPPRESSOR OF ddc (SDC).

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

In one embodiment, the nucleic acid silencer molecule of the present invention comprises a promoter region of a plant endogene target, i.e., a plant endogene to be downregulated via TGS. The nucleic acid silencer molecule of this embodiment encodes either a single-stranded silencer or an inverted repeat (IR) silencer, either of which is initially an RNA molecule that is transcribed from the nucleic acid construct. The single-stranded silencer and IR silencer provide TGS of endogenes in plants, plant tissues and plant cells. In one embodiment, the single-stranded silencer is an RNA molecule that is produced from a promoter region of a plant endogene target (i.e., a nucleic acid silencer molecule) that is in an antisense orientation with respect to the plant operable promoter in the nucleic acid construct. In another embodiment, the single-stranded silencer is an RNA molecule that is produced from a promoter region of a plant endogene target (i.e., a nucleic acid silencer molecule) that is in a sense orientation with respect to the plant operable promoter in the nucleic acid construct. In each of these single-stranded silencer embodiments, the construct, nucleic acid silencer molecule and single-stranded silencers are in the absence of inverted repeat structures, i.e., no inverted repeat structures or inverted repeats are present in the nucleic acid construct and products produced from it. Alternatively, in another embodiment, the nucleic acid silencer molecule is an inverted repeat silencer, and is an RNA molecule that is produced from a promoter region of a plant endogene target, wherein the RNA molecule is provided in duplicate and arranged in an inverted configuration in the nucleic acid construct. In one embodiment, the duplicate copies of the target sequence in the inverted repeat structure are separated by a spacer. In one embodiment, the spacer contains an intron functional in a plant cell. In another embodiment, the spacer is a fragment from a soybean 7S promoter. However, it is contemplated that the spacer sequence is not limited to these features and may be any sequence suitable for allowing the inverted repeat sequences to hybridize. Such a spacer sequence is exemplified by SEQ ID NO:15. In some embodiments, expression of the nucleic acid silencer molecule produces an initial single stranded RNA. This single stranded RNA may be converted to a double stranded RNA by cellular mechanisms or as a result of the inverted repeat structure.

In one embodiment, the promoter region comprises nucleotides upstream of the transcription start site of the target gene. In another embodiment, the promoter region comprises nucleotides upstream of the transcription start site and nucleotides downstream of the transcription start site of the target gene. In some embodiments, the promoter region comprises about 300 nucleotides to about 1500 nucleotides. In other embodiments, the promoter region comprises about 400 nucleotides to about 1200 nucleotides. In additional embodiments, the promoter region comprises about 425 nucleotides to about 1100 nucleotides. In further embodiments, the promoter region comprises about 425 nucleotides to about 1075 nucleotides. See U.S. provisional patent application Ser. No. 61/698,203, filed 7 Sep. 2012, incorporated herein by reference in its entirety.

In one embodiment, the nucleic acid silencer molecule of the present invention comprises a promoter region of a plant endogene target, i.e., a plant endogene to be downregulated via TGS. In another embodiment, the nucleic acid silencer molecule comprise the 2000 nucleotides immediately 5′ to the transcription start site of the endogene to be downregulated, or a fragment of this 2000 nucleotide region. The fragment may comprise at least 100, 200, 300, 400, 500, 600, 700, 800, 900, 1000, 1100, 1200, 1300, 1400, 1500, 1600, 1700, 1800, 1900 or 2000 contiguous nucleotides of the region that is 2000 nucleotides immediately 5′ to the transcription start site of the endogene to be downregulated.

In one embodiment, sRNAs are produced from the expressed nucleic acid silencer molecule in a cell containing the nucleic acid silencer molecule. In another embodiment, the silencing enhancer enhances the production of sRNAs from the expressed nucleic acid silencer molecule in a cell containing the silencing enhancer operatively linked to the nucleic acid silencer molecule.

Any promoter that is operable in a plant may be used in the nucleic acid construct. In some embodiments, the promoter is a single copy of a plant operable promoter, including those described herein. In other embodiments, the promoter is a double copy of a plant operable promoter to make a homologous double promoter. In further embodiments, the promoter is a combination of two different promoters to make a heterologous double promoter. In one embodiment, the plant operable promoter is a double 35S CMV promoter. In an additional embodiment, the plant operable promoter is a single 35S CMV promoter. The sequence of the double 35S CMV promoter is set forth in SEQ ID NO:16. The nucleic acid construct may further comprise sequences to enable cloning of the nucleic acid construct or sequences that will facilitate splicing. In one embodiment, the additional sequence may be a 3′ sequence that is operable in plants. In another embodiment, the 3′ sequence is derived from TRV2 (tobacco rattle virus 2) which is positioned downstream of the nucleic acid silencer molecule. The sequence of the TRV2 3′ sequence is set forth in SEQ ID NO:17.

The nucleic acid construct may also comprise plant operable 3′ regulatory sequences. In one embodiment, the plant operable 3′ regulatory sequence is a polyA addition sequence. In another embodiment the polyA addition sequence is a NOS polyA sequence.

In a second aspect, the present invention provides a transgenic plant cell comprising the nucleic acid construct. In one embodiment, the nucleic acid construct is stably integrated into the genome of the transgenic plant cell. The transgenic plant cell is prepared by transfecting a plant cell with a nucleic acid construct using methods well known in the art including, but not limited to, those described herein. Plant cells of a wide variety of plant species can be transfected with a nucleic acid construct of the present invention. A plant cell containing the nucleic acid construct is selected in accordance with conventional techniques including, but not limited to, those described herein. The plant cell is grown under conditions suitable for the expression of the nucleic acid in the transfected plant cell using growth conditions well known in the art.

The present invention may be used for transfecting plant cells of a wide variety of plant species, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setara italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats (Avena sativa), barley (Hordeum vulgare), switchgrass (Panicum virgatum), vegetables, ornamentals, and conifers. See U.S. Pat. No. 7,763,773 for a list of additional plant species that can be used in accordance with the present invention.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulchernima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesil); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

In a third aspect, the present invention provides a transgenic plant comprising the nucleic acid construct. In one embodiment, the nucleic acid construct is stably integrated into the genome of the transgenic plant. Transgenic plants are regenerated from transgenic plant cells described herein using conventional techniques well known to the skilled artisan using various pathways, including somatic embryogenesis and organogenesis. Transformed plant cells which are derived by plant transformation techniques, including those discussed above, can be cultured to regenerate a whole plant which possesses the transformed genotype, and thus the desired phenotype. Such regeneration techniques generally rely on manipulation of certain phytohormones in a tissue culture growth medium, typically relying on a marker which has been introduced together with the desired nucleotide sequences. See, for example, U.S. Pat. No. 7,763,773, U.S. Patent Application Publication No. 2010/0199371 and International Published Application No. WO 2008/094127 and references cited therein. The transgenic plant is grown under conditions suitable for the expression of the nucleic acid in the transfected plant using growth conditions well known in the art.

In a fourth aspect, the present invention provides a method of more effectively silencing a gene of interest, such as more effectively silencing endogenous gene expression, in plants, plant tissues or plant cells by transcriptional gene silencing. In one embodiment, the method comprises transfecting a plant cell with a nucleic acid construct to produce a transgenic plant cell as described herein. The method further comprises expressing the nucleic acid silencer molecule described herein in the transgenic plant cell as described herein. The expressed nucleic acid silencer molecule described herein is cleaved in the transgenic plant cell to produce one or more small RNAs (sRNAs) that induces transcriptional gene silencing to reduce expression of the target gene of interest. In some embodiments, expression of the nucleic acid silencer molecule produces an initial single stranded RNA. This single stranded RNA may be converted to a double stranded RNA before processing to produce sRNAs by cellular mechanisms or as a result of the inverted repeat structure. The method may optionally include preparing a nucleic acid construct encoding a nucleic acid as described herein. In another embodiment, the method comprises regenerating a transgenic plant from the transgenic plant cell. In this embodiment, the nucleic acid silencer molecule is expressed in the transgenic plant. The expressed nucleic acid is cleaved in the transgenic plant cell to produce one or more sRNAs that induces transcriptional gene silencing to reduce expression of the target gene of interest.

The nucleic acid molecule comprising a silencing enhancer as described herein and a nucleic acid silencing molecule as described herein that is inserted into plants (nucleic acid molecule of interest) in accordance with the present invention is not critical to the transformation process. Generally the nucleic acid molecule of interest that is introduced into a plant is part of a construct as described herein. The construct typically includes regulatory regions operatively linked to the 5′ side of the nucleic acid molecule of interest and/or to the 3′ side of the nucleic acid molecule of interest. A cassette containing all of these elements is also referred to herein as an expression cassette. The expression cassettes may additionally contain 5′ leader sequences in the expression cassette construct. The regulatory regions (i.e., promoters, transcriptional regulatory regions, and transcriptional termination regions) may be native/analogous to the host cell or to each other. Alternatively, the regulatory regions may be heterologous to the host cell or to each other. See, U.S. Pat. Nos. 7,205,453 and 7,763,773, and U.S. Patent Application Publication Nos. 2006/0218670, 2006/0248616 and 20090100536, and the references cited therein.

The nucleic acid molecule of interest that is under control of a plant operable promoter may be any nucleic acid molecule as defined herein and may be used to alter any characteristic or trait of a plant species into which it is introduced through the mechanism of transcriptional gene silencing in order to downregulate the target gene. The target gene may encode a regulatory protein, such as a transcription factor and the like, a binding or interacting protein, or a protein that alters a phenotypic trait of a transgenic plant cell or a transgenic plant. Downregulation of a target gene may enhance, alter or otherwise modify a trait of the plant, such as an agronomic trait. The agronomic trait may relate to plant morphology, physiology, growth and development, yield, nutrition, disease or pest resistance, or environmental or chemical tolerance. In some aspects, the trait is selected from group of traits consisting of water use efficiency, temperature tolerance, yield, nitrogen use efficiency, seed protein, seed oil and biomass. Yield may include increased yield under non-stress conditions and increased yield under environmental stress conditions. Stress conditions may include, for example, drought, shade, fungal disease, viral disease, bacterial disease, insect infestation, nematode infestation, extreme temperature exposure (cold or hot), osmotic stress, reduced nitrogen nutrient availability, reduced phosphorus nutrient availability and high plant density. In some embodiments, the nucleic acid molecule of interest may be used to modify metabolic pathways, such as fatty acid biosynthesis or lipid biosynthesis pathways in seeds, or to modify resistance to pathogens in plants.

Generally, the expression cassette may additionally comprise a selectable marker gene for the selection of transformed cells. Selectable marker genes are utilized for the selection of transformed cells or tissues. Usually, the plant selectable marker gene will encode antibiotic resistance, with suitable genes including at least one set of genes coding for resistance to the antibiotic spectinomycin, the streptomycin phosphotransferase (spt) gene coding for streptomycin resistance, the neomycin phosphotransferase (nptII) gene encoding kanamycin or geneticin resistance, the hygromycin phosphotransferase (hpt or aphiv) gene encoding resistance to hygromycin, acetolactate synthase (als) genes. Alternatively, the plant selectable marker gene will encode herbicide resistance such as resistance to the sulfonylurea-type herbicides, glufosinate, glyphosate, ammonium, bromoxynil, imidazolinones, and 2,4-dichlorophenoxyacetate (2,4-D), including genes coding for resistance to herbicides which act to inhibit the action of glutamine synthase such as phosphinothricin or Basta (e.g., the bar gene). See generally, International Publication No. WO 02/36782, U.S. Pat. Nos. 7,205,453 and 7,763,773, and U.S. Patent Application Publication Nos. 2006/0218670, 2006/0248616, 2007/0143880 and 20090100536, and the references cited therein. See also, Jefferson et al. (1991); De Wet et al. (1987); Goff et al. (1990); Kain et al. (1995) and Chiu et al. (1996). This list of selectable marker genes is not meant to be limiting. Any selectable marker gene can be used. The selectable marker gene is also under control of a promoter operable in the plant species to be transformed. Such promoters include those described in International Publication No. WO 2008/094127 and the references cited therein. See also, U.S. Patent Application Publication Nos. 2008/0313773 and 2010/0199371 for an exemplification of additional markers that can be used in accordance with the present invention.

A number of promoters can be used in the practice of the invention. The promoters can be selected based on the desired outcome. That is, the nucleic acids can be combined with constitutive, tissue-preferred, or other promoters for expression in the host cell of interest. Such constitutive promoters include, for example, the core promoter of the Rsyn7 (WO 99/48338 and U.S. Pat. No. 6,072,050); the core CaMV35S promoter (Odell et al., 1985); rice actin (McElroy et al., 1990); ubiquitin (Christensen and Quail, 1989; Christensen et al., 1992); pEMU (Last et al., 1991); MAS (Velten et al., 1984); ALS promoter (U.S. Pat. No. 5,659,026), and the like. Other constitutive promoters include, for example, those disclosed in U.S. Pat. Nos. 5,608,149; 5,608,144; 5,604,121; 5,569,597; 5,466,785; 5,399,680; 5,268,463; and 5,608,142.

Other promoters include inducible promoters. Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners Pathogen-inducible promoters include those from pathogenesis-related proteins (PR proteins), which are induced following infection by a pathogen; e.g., PR proteins, SAR proteins, beta-1,3-glucanase, chitinase, etc. Other promoters include those that are expressed locally at or near the site of pathogen infection. In further embodiments, the promoter may be a wound-inducible promoter. In other embodiments, chemical-regulated promoters can be used to modulate the expression of a gene in a plant through the application of an exogenous chemical regulator. The promoter may be a chemical-inducible promoter, where application of the chemical induces gene expression, or a chemical-repressible promoter, where application of the chemical represses gene expression. In addition, tissue-preferred promoters can be utilized to target enhanced expression of a polynucleotide of interest within a particular plant tissue. Each of these promoters is described in U.S. Pat. Nos. 6,506,962, 6,575,814, 6,972,349 and 7,301,069 and in U.S. Patent Application Publication Nos. 2007/0061917 and 2007/0143880. See also, U.S. Patent Application Publication Nos. 2008/0313773 and 2010/0199371 for an exemplification of additional promoters that can be used in accordance with the present invention. Other promoters known to the skilled artisan to be useful in plants, can also be used in the present invention.

Promoters for use in the current invention may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (U.S. Patent Application Publication No. 2006/0156439), the maize ROOTMET2 promoter (International Publication No. WO 05/063998), the CR1BIO promoter (International Publication No. WO 06/055487), the CRWAQ81 promoter (International Publication No. WO 05/035770) and the maize ZRP2.47 promoter (NCBI accession number: U38790; GI No. 1063664). In some embodiments, the promoter that is used is a double promoter, for example, a double CaMV 35S promoter. Double promoters of any of the promoters disclosed herein, as well as other promoters known to the skilled artisan to be useful in plants, can be used in the present invention.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g. transitions and transversions may be involved.

Once a nucleic acid has been cloned into an expression vector, it may be introduced into a plant cell using conventional transformation (or transfection) procedures. The term “plant cell” is intended to encompass any cell derived from a plant including undifferentiated tissues such as callus and suspension cultures, as well as plant seeds, pollen or plant embryos. Plant tissues suitable for transformation include leaf tissues, root tissues, meristems, protoplasts, hypocotyls, cotyledons, scutellum, shoot apex, root, immature embryo, pollen, and anther. “Transformation” means the directed modification of the genome of a cell by the external application of recombinant DNA, leading to its uptake and integration into the subject cell's genome. In this manner, genetically modified plants, plant cells, plant tissue, seed, and the like can be obtained.

DNA constructs in accordance with the present invention can be used to transform any plant. The constructs may be introduced into the genome of the desired plant host by a variety of conventional techniques. Techniques for transforming a wide variety of higher plant species are well known and described in the technical and scientific literature. Transformation protocols may vary depending on the type of plant or plant cell, i.e., monocot or dicot, targeted for transformation, as is well known to the skilled artisan. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Thus, any method, which provides for effective transformation/transfection may be employed. See, for example, U.S. Pat. Nos. 7,241,937, 7,273,966 and 7,291,765 and U.S. Patent Application Publication Nos. 2007/0231905 and 2008/0010704 and references cited therein. See also, International Published Application Nos. WO 2005/103271 and WO 2008/094127 and references cited therein. See also, U.S. Patent Application Publication Nos. 2008/0313773 and 2010/0199371 for an exemplification of transformation protocols for a variety of plant species that can be used in accordance with the present invention.

Transformed plant cells which are derived by any of the above transformation techniques can be cultured to regenerate a whole plant which possesses the transformed genotype and thus the desired phenotype, e.g., a transgenic plant. A “transgenic plant” is a plant into which foreign DNA has been introduced. A “transgenic plant” encompasses all descendants, hybrids, and crosses thereof, whether reproduced sexually or asexually, and which continue to harbor the foreign DNA. Regeneration techniques rely on manipulation of certain phytohormones in a tissue culture growth medium, typically relying on a biocide and/or herbicide marker which has been introduced together with the desired nucleotide sequences. See for example, International Published Application No. WO 2008/094127 and references cited therein and U.S. Patent Application Publication No. 2010/0199371.

The foregoing methods for transformation are typically used for producing a transgenic variety in which the expression cassette is stably incorporated. After the expression cassette is stably incorporated in transgenic plants, it can be transferred to other plants by sexual crossing. In one embodiment, the transgenic variety could then be crossed, with another (non-transformed or transformed) variety, in order to produce a new transgenic variety. Alternatively, a genetic trait which has been engineered into a particular cotton line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties which do not contain that gene. As used herein, “crossing” can refer to a simple X by Y cross, or the process of backcrossing, depending on the context. Any of a number of standard breeding techniques can be used, depending upon the species to be crossed.

Once transgenic plants of this type are produced, the plants themselves can be cultivated in accordance with conventional procedures. Transgenic seeds can, of course, be recovered from the transgenic plants. These seeds can then be planted in the soil and cultivated using conventional procedures to produce transgenic plants. The cultivated transgenic plants will express the nucleic acid as described herein and it will be cleaved to produce sRNAs.

In a fifth aspect, the present invention provides nucleic acid constructs and methods to identify and obtain other silencing enhancers of plant TGS. According to this aspect, the nucleic acid construct is one that is suitable for transformation of a plant species for which it is desired to identify a silencing enhancer. In one embodiment, the nucleic acid construct may be included in a vector. In some embodiments, the vector is suitable for Agrobacterium-mediated transformation. In other embodiments, the vector may be suitable for biolistic-mediated transformation. Other suitable vectors for plant transformations are well known to the skilled artisan. In another embodiment, the nucleic acid construct may be used directly for the transformation of a plant according to techniques well known to the skilled artisan. The nucleic acid construct comprises a plant operable promoter operatively linked to a putative silencing enhancer operatively linked to a nucleic acid silencer molecule described herein operatively linked to plant operable 3′ regulatory region.

In one embodiment, the polynucleotide to be tested for silencing enhancer activity is a region of the promoter of a homolog of SUPPRESSOR OF ddc (SDC) from a species other than Arabidopsis. The homolog of SDC may be from a monocot or a dicot. The homolog of SDC may be selected from the group consisting of: maize, soybean, sunflower, sorghum, canola, wheat, alfalfa, cotton, rice, barley, millet, sugar cane and switchgrass. In another embodiment, the putative silencing enhancer or polynucleotide which is to be tested for enhancing transcriptional gene silencing is a promoter region of a gene loci that is silenced by higher levels of endogenous 24 nt siRNAs and is heavily methylated. In one embodiment, the plant operable promoter is a single promoter, a double homologous promoter or a double heterologous promoter. In one embodiment, the plant operable promoter is a single or a double 35S CMV promoter. In one embodiment, the 3′ sequence is a TRV2 3′ sequence. In an additional embodiment, the 3′ regulatory region is a polyA addition sequence. In one embodiment, the polyA sequence is a NOS poly A sequence. The testing for more effective silencing may be carried out according to methods well known in the art. These methods include, but are not limited to, RT-PCR, PCR, northern blot analysis, immunological assay and enzymatic assay. In some embodiments, the methylation state of the target promoter may be assayed by McrBc enzymatic digestion. In other embodiments, the state of chromatin modification is tested by immunological assays utilizing antibodies directed to sites of histone methylation or acetylation. In further embodiments, the amount of gene product is determined. The testing of fragments of the promoter region to be tested for more effectively silencing a gene of interest may be carried out according to methods described herein.

Further according to this aspect, suitable silencing enhancers are identified by a method which comprises the steps of preparing a nucleic acid construct comprising a putative silencing enhancer and a nucleic acid silencer molecule as described herein, transforming a cell or tissue of a plant species of interest with the nucleic acid construct and determining whether the putative silencing enhancer more effectively silences the test endogene. If the silencing of the test endogene is more effectively silenced, then the putative silencing enhancer is identified as a silencing enhancer of TGS. In one embodiment, the determination is made by culturing the transformed plant cell for expression of the nucleic acid silencer molecule and testing for transcriptional gene silencing in the cultured transformed plant cell or tissue. In another embodiment, the determination is made by regenerating a transformed plant from the transformed plant cell or tissue and testing for transcriptional gene silencing in the transformed plant. The regeneration of transformed plants is performed according to techniques well known to the skilled artisan. The testing for silencing may be carried out according to methods well known in the art. These methods include, but are not limited to, RT-PCR, PCR, northern blot analysis, immunological assay and enzymatic assay, as well as other methods described herein.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, New York); Sambrook et al., 1989, Molecular Cloning, 2nd Ed. (Cold Spring Harbor Laboratory Press, Cold Spring Harbor, New York); Sambrook and Russell, 2001, Molecular Cloning, 3rd Ed. (Cold Spring Harbor Laboratory Press, Cold Spring Harbor, New York); Green and Sambrook, 2012, Molecular Cloning, 4th Ed. (Cold Spring Harbor Laboratory Press, Cold Spring Harbor, New York); Ausubel et al., 1992), Current Protocols in Molecular Biology (John Wiley & Sons, including periodic updates); Glover, 1985, DNA Cloning (IRL Press, Oxford); Russell, 1984, Molecular biology of plants: a laboratory course manual (Cold Spring Harbor Laboratory Press, Cold Spring Harbor, N.Y.); Anand, Techniques for the Analysis of Complex Genomes, (Academic Press, New York, 1992); Guthrie and Fink, Guide to Yeast Genetics and Molecular Biology (Academic Press, New York, 1991); Harlow and Lane, 1988, Antibodies, (Cold Spring Harbor Laboratory Press, Cold Spring Harbor, New York); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Fire et al., RNA Interference Technology: From Basic Science to Drug Development, Cambridge University Press, Cambridge, 2005; Schepers, RNA Interference in Practice, Wiley-VCH, 2005; Engelke, RNA Interference (RNAi): The Nuts & Bolts of siRNA Technology, DNA Press, 2003; Gott, RNA Interference, Editing, and Modification: Methods and Protocols (Methods in Molecular Biology), Human Press, Totowa, N.J., 2004; Sohail, Gene Silencing by RNA Interference: Technology and Application, CRC, 2004.

### EXAMPLES

The present invention is described by reference to the following Examples, which is offered by way of illustration and is not intended to limit the invention in any manner. Standard techniques well known in the art or the techniques specifically described below were utilized.

### Example 1

### Vector Construction

Using standard PCR and cloning techniques, a 1,731-bp Arabidopsis thaniana wild type (WT) genomic DNA fragment from the promoter region (position −274 to −2,004; transcription start site is +1) of AT2G17690 (SDC) was cloned into a Gateway entry vector to generate pENTR/PSDC. A 475-bp genomic DNA fragment from the promoter region (-9 to −483; transcription start site is +1) of AT1G80080 (TMM) was cloned into a Gateway entry vector to give pENTR/PTMM-S. The primers used to clone these two genome DNA fragments contained the restriction enzyme sites, AvrII in the forward primer and SpeI, AscI, in the reverse primer. pENTR/PSDC was double digested by SpeI and AscI to linearized pENTR/PSDC. pENTR/PTMM-S was digested by AvrII and AscI to release PTMM-S DNA fragment. Because of the compatible digested ends of AvrII and SpeI, the digested PTMM-S DNA fragment could be ligated into the linear pENTR/PSDC to generate pENTR/PSDC-PTMM-S. pENTR/PTMM-S and pENTR/PSDC-PTMM-S were cloned into pBADC, a Gateway destination vector modified from pBA002 (Kost et al., 1998), by Gateway LR reaction to generate pBA/PTMM-S and pBA/PSDC-PTMM-S respectively. In both constructs the sequences were placed downstream of a CaMV 35S promoter. These two entry vectors were also cloned into pKGWFS7 to give pKGWFS7/PTMM-S and pKGWFS7/PSDC-PTMM-S where no promoter was placed upstream of the sequences. By this method, the following vectors (pBA/PSDC-PTMM-S, pBA/PSDC2-PTMM-S, pBA/PSDC3-PTMM-S, pBA/PSDC4-PTMM-S, pBA/PSDC5-PTMM-S, pBA/PSDC6-PTMM-S, pBA/PSDC7-PTMM-S, pBA/PSDC8-PTMM-S, pBA/PTMM-AS, pBA/PSDC-PTMM-AS, pBA/PSDC3-PTMM-AS, pBA/PSDC7-PTMM-AS, pBA/PTMM-IR, pBA/PSDC3-PTMM-IR, pBA/PSDC7-PTMM-IR, pBA/PFAD2, pBA/PSDC3-PFAD2, pBA/PCH42, pBA/PSDC4-PCH42, pBA/TMM-S, pBA/PSDC3-TMM-S) were constructed and in all these constructs a CaMV promoter was used to transcribe the downstream sequences. These DNA sequences and primers used to clone these DNA fragments are given below.

PSDC:
(SEQ ID NO: 1)
gaaaatgaggaaagtgttgtgccaaatcacgaattagtacccatgtctgt
tgatgctctaacaattctttccacaagaccacaactccacaagtaacccg
cgctatgttttcacgggtatcgacaatgttctcaggttccactataaact
cattatccattgagaatttagtgcgcagaggagttgcttcaagttgattg
accaatacttttttcaattgttagacataaacactgaatacaaatctttg
aacaacctgacttagatcatcctcattgcacaataaaacacagttttttt
tttttttttttttaaatgaaaaataaaataaaatgttatttagtgctttt
aggaggaagactcactgtctttcatgtctctgcaaagacattttcttact
tttaaagacatcacacttgactaataaatgactcaaattaaaataatgtt
aatttttataataaactataataaagacatctctaaaaaagtcaccaatg
agaatgctcttaggaaccaaattttgcatgaggaaaactgttagctaagt
tagggtctggtttagaactcaagtccaactttcaaaatttaaaagaacta
acttagtcaggttacgggttcttgggaatacgtttgtagtcgaaagaact
gttttctcgatcgaaatttttggtaaaacatgaatgaaagaaaggtgatc
tccgacgaaaaattaataacctaatttatcatgaggttttgtagactaat
tttcttttactaaagtagtgtatttaagactcggtaatcaattaactatg
ttcagaatttttggttgtttggattgacttttgggtaaaagaatttagaa
ttgcaaagaattgcaaacaatcggtttgttcggattaggtaaaagcattc
cgaattgaaaacctaagtttgtcttggacccctaaaatttaagcttaatt
tatcaatttcggtacatttgcaaagttcagtccaattcaaatattcggtt
tagaccaaaagttcgaataatgaaaaaaaaacatctgaaagttgtggaat
ttttttttaaagcaaattggtaaaccataagaacttctttaaaagtccac
cacatcatttatccttcaaaatctccccaaatacgaaatcaagcaagtct
tccaaaatttagaatctccaatacaccatgttgggaaacaaaccaacttt
ttacatccgaagattatagatatattgtctaatatatcaatcaagtccaa
ctccaattagaagaaagtttttttggaaaaaagccctaaacaaatttgtg
caagttttaccacttggacccaaagcggacaatatcatcctaatcgaaaa
agttggaatgggcttggagagcccaacaaccctattttttagaaaacaat
aaatttatttgcaaaacgacatgaaaatcatcgtacaaaacattcaagat
atatgatgaattatttgattattaccacgtcagtagttatgaagataaga
ttttacagtacacgttgatataaagatgagatttcgctgtacacgtcagt
tatttagataaaatttcacagtacacgtcagttataaagataagatttca
cagtacacatcagttataaagataagatttcacagtacacgtcagttata
aagataagatttcattgtacacgttagttataaagataagatttcacaat
acacgtcagccctaacacaaaacatactag (1731 bp).
PTMM-S:
(SEQ ID NO: 2)
tgttgctccatgggcatgtgcttttcgtatgcacagaccacgcgtccgtt
tattcagattctcgtgttacaacaaaataatgattttaaggttcagaatc
aaacatatgaaactttttatcaattgctatctacggtttgttacaaaaac
aaatacagtgcccagttcaaaatacaaattgaataatagcccaacatatc
gtaaatgggaaaatgggctcagcccaaataaaaaaaaagtaaacgataag
gagacgtcgggtcccgttcacgaagcggtcggatctagggcccaacacaa
gacgaatgaagggaatatctcgtgaaagcaataaatgcagaaatcgagaa
tcatgaattttatttatttcgctcctcgagttatcccgtgaaagtcataa
agacattgcgaacgtttttatggacccaccaattgtttcttcagaattat
acatatacacactgacaatttcgaa (475 bp).
PSDC1:
(SEQ ID NO: 3)
gaaaatgaggaaagtgttgtgccaaatcacgaattagtacccatgtctgt
tgatgctctaacaattctttccacaagaccacaactccacaagtaacccg
cgctatgttttcacgggtatcgacaatgttctcaggttccactataaact
cattatccattgagaatttagtgcgcagaggagttgcttcaagttgattg
accaatacttttttcaattgttagacataaacactgaatacaaatctttg
aacaacctgacttagatcatcctcattgcacaataaaacacagttttttt
tttttttttttttaaatgaaaaataaaataaaatgttatttagtgctttt
aggaggaagactcactgtctttcatgtctctgcaaagacattttcttact
tttaaagacatcacacttgactaataaatgactcaaattaaaataatgtt
aatttttataataaactataataaagacatctctaaaaaagtcaccaatg
agaatgctcttaggaaccaaattttgcatgaggaaaactgttagctaagt
tagggtctggtttagaactcaagtccaactttcaaaatttaaaagaacta
acttagtcaggttacgggttcttgggaatacgtttgtagtcgaaagaact
gttttctcgatcgaaatttttggtaaaacatgaatgaaagaaaggtgatc
tccgacgaaaaattaataacctaatttatcatgaggttttgtagactaat
tttctttttactaaagtagtgtatttaagactcggtaatcaattaactat
gttcagaatttttggttgtttggattgacttttgggtaaaagaatttaga
attgcaaagaattgcaaacaatcggtttgttcggattaggtaaaagcatt
ccgaattgaaaacctaagtttgtcttggacccctaaaatttaagcttaat
ttatcaatttcggtacatttgcaaagttcagtccaattcaaatattcggt
ttagaccaaaagttcgaataatgaaaaaaaaacatctgaaagttgtggaa
tttttttttaaagcaaattggtaaaccataagaacttctttaaaagtcca
ccacatcatttatccttcaaaatctccccaaatacgaaatcaagcaagtc
ttccgaaatttagaatctccaatacaccatgttgggaaacaaaccaactt
tttacatccgaagattatagatatattgtctaatatatcaatcaagtcca
actccaattagaagaaagtttttttggaaaaaagccctaaacaaatttgt
gcaagttttaccacttggacccaaagcggacaatatcatcctaatcgaaa
aagttggaatgggcttggagagcccaacaaccctattttttagaaaacaa
taaatttatttgcaaaacgacatgaaaatcatcgtacannncattcaaga
tatatgatgaattatt (1466 bp).
PSDC2:
(SEQ ID NO: 4)
gaaaatgaggaaagtgttgtgccaaatcacgaattagtacccatgtctgt
tgatgctctaacaattctttccacaagaccacaactccacaagtaacccg
cgctatgttttcacgggtatcgacaatgttctcaggttccactataaact
cattatccattgagaatttagtgcgcagaggagttgcttcaagttgattg
accaatacttttttcaattgttagacataaacactgaatacaaatctttg
aacaacctgacttagatcatcctcattgcacaataaaacacagtattttt
ttttttttttttaaatgaaaaataaaataaaatgttatttagtgctttta
ggaggaagactcactgtctttcatgtctctgcaaagacattttcttactt
ttaaagacatcacacttgactaataaatgactcaaattaaaataatgtta
atttttataataaactataataaagacatctctaaaaaagtcaccaatga
gaatgctcttaggaaccaaattttgcatgaggaaaactgttagctaagtt
agggtctggtttagaactcaagtccaactttcaaaatttaaaagaactaa
cttagtcaggttacgggttcttgggaatacgtttgtagtcgaaagaactg
ttttctcgatcgaaatttttggtaaaacatgaatgaaagaaaggtgatct
ccgacgaaaaattaataacctaatttatcatgaggttttgtagactaatt
ttctttttactaaagtagtgtatttaagactcggtaatcaattaactatg
ttcagaatttttggttgtttggattgacttttgggtaaaagaatttagaa
ttgcaaagaattgcaaacaatcggtttgttcggattaggtaaaagcattc
cgaattgaaaacctaagtttgtcttggacccctaaaatttaagcttaatt
tatcaatttcggtacatttgcaaagttcagtccaattcaaatattcggtt
tagaccaaaagttcgaataatgaaaaaaaaacatctgaaagttgtggaat
ttttttttaaagcaaattggtaaaccataagaacttctttaaaagtccac
cacatcatttatccttcaaaatctccccaaatacgaaatcaagcaagtct
tccaaaatttagaatctccaatacaccat (1179 bp).
PSDC3:
(SEQ ID NO: 5)
gaaaatgaggaaagtgttgtgccaaatcacgaattagtacccatgtctgt
tgatgctctaacaattctttccacaagaccacaactccacaagtaacccg
cgctatgttttcacgggtatcgacaatgttctcaggttccactataaact
cattatccattgagaatttagtgcgcagaggagttgcttcaagttgattg
accaatacttttttcaattgttagacataaacactgaatacaaatctttg
aacaacctgacttagatcatcctcattgcacaataaaacacagttttttt
tttttttttttttaaatgaaaaataaaataaaatgttatttagtgctttt
aggaggaagactcactgtctttcatgtctctgcaaagacattttcttact
tttaaagacatcacacttgactaataaatgactcaaattaaaataatgtt
aatttttataataaactataataaagacatctctaaaaaagtcaccaatg
agaatgctcttaggaaccaaattttgcatgaggaaaactgttagctaagt
tagggtctggtttagaactcaagtccaactttcaaaatttaaaagaacta
acttagtcaggttacgggttcttgggaatacgtttgtagtcgaaagaact
gttttctcgatcgaaatttttggtaaaacatgaatgaaagaaaggtgatc
tccgacgaa (709 bp).
PSDC4:
(SEQ ID NO: 6)
gaaaatgaggaaagtgttgtgccaaatcacgaattagtacccatgtctgt
tgatgctctaacaattctttccacaagaccacaactccacaagtaacccg
cgctatgttttcacgggtatcgacaatgttctcaggttccactataaact
cattatccattgagaatttagtgcgcagaggagttgcttcaagttgattg
accaatacttttttcaattgttagacataaacactgaatacaaatctttg
aacaacctgacttagatcatcctcattgcacaataaaacacagttttttt
tttttttttttttaaatgaaaaataaaataaaatgttatttagtgctttt
aggaggaagactcactgtctttcatgtctctgcaaagac (389 bp).
PSDC5:
(SEQ ID NO: 7)
attttcttacttttaaagacatcacacttgactaataaatgactcaaatt
aaaataatgttaatttttataataaactataataaagacatctctaaaaa
agtcaccaatgagaatgctcttaggaaccaaattttgcatgaggaaaact
gttagctaagttagggtctggtttagaactcaagtccaactttcaaaatt
taaaagaactaacttagtcaggttacgggttcttgggaatacgtttgtag
tcgaaagaactgttttctcgatcgaaatttttggtaaaacatgaatgaaa
gaaaggtgatctccgacgaaaaattaataacctaatttatcatgaggttt
tgtagactaattttctttttactaaagtagtgtatttaagactcggtaat
caattaactatgttcagaatttttggttgtttggattgacttttgggtaa
aagaatttagaattgcaaagaattgcaaacaatcggtttgttcggattag
gtaaaagcattccgaattgaaaacctaagtttgtcttggacccctaaaat
ttaagcttaatttatcaatttcggtacatttgcaaagttcagtccaattc
aaatattcggtttagaccaaaagttcgaataatgaaaaaaaaacatctga
aagttgtggaatttttttttaaagcaaattggtaaaccataagaacttct
ttaaaagtccaccacatcatttatccttcaaaatctccccaaatacgaaa
tcaagcaagtcttccaaaatttagaatctccaatacaccatgttgggaaa
caaaccaactttttacatccgaagattatagatatattgtctaatatatc
aatcaagtccaactccaattagaagaaagtttttttggaaaaaagcccta
aacaaatttgtgcaagttttaccacttggacccaaagcggacaatatcat
cctaatcgaaaaagttggaatgggcttggagagcccaacaaccctatttt
ttagaaaacaataaatttatttgcaaaacgacatgaaaatcatcgtacaa
aacattcaagatatatgatgaattatttgattattaccacgtcagtagtt
atgaagataagattttacagtacacgttgatataaagatgagatttcgct
gtacacgtcagttatttagataaaatttcacagtacacgtcagttataaa
gataagatttcacagtacacatcagttataaagataagatttcacagtac
acgtcagttataaagataagatttcattgtacacgttagttataaagata
agatttcacaatacacgtcagccctaacacaaaacatactag
(1342 bp).
PSDC6:
(SEQ ID NO: 8)
aaattaataacctaatttatcatgaggttttgtagactaattttcttttt
actaaagtagtgtatttaagactcggtaatcaattaactatgttcagaat
ttttggttgtttggattgacttttgggtaaaagaatttagaattgcaaag
aattgcaaacaatcggtttgttcggattaggtaaaagcattccgaattga
aaacctaagtttgtcttggacccctaaaatttaagcttaatttatcaatt
tcggtacatttgcaaagttcagtccaattcaaatattcggtttagaccaa
aagttcgaataatgaaaaaaaaacatctgaaagttgtggaattttttttt
aaagcaaattggtaaaccataagaacttctttaaaagtccaccacatcat
ttatccttcaaaatctccccaaatacgaaatcaagcaagtcttccaaaat
ttagaatctccaatacaccatgttgggaaacaaaccaactttttacatcc
gaagattatagatatattgtctaatatatcaatcaagtccaactccaatt
agaagaaagtttttttggaaaaaagccctaaacaaatttgtgcaagtttt
accacttggacccaaagcggacaatatcatcctaatcgaaaaagttggaa
tgggcttggagagcccaacaaccctattttttagaaaacaataaatttat
ttgcaaaacgacatgaaaatcatcgtacaaaacattcaagatatatgatg
aattatttgattattaccacgtcagtagttatgaagataagattttacag
tacacgttgatataaagatgagatttcgctgtacacgtcagttatttaga
taaaatttcacagtacacgtcagttataaagataagatttcacagtacac
atcagttataaagataagatttcacagtacacgtcagttataaagataag
atttcattgtacacgttagttataaagataagatttcacaatacacgtca
gccctaacacaaaacatactag (1022 bp).
PSDC7:
(SEQ ID NO: 9)
gttgggaaacaaaccaactttttacatccgaagattatagatatattgtc
taatatatcaatcaagtccaactccaattagaagaaagtttttttggaaa
aaagccctaaacaaatttgtgcaagttttaccacttggacccaaagcgga
caatatcatcctaatcgaaaaagttggaatgggcttggagagcccaacaa
ccctattttttagaaaacaataaatttatttgcaaaacgacatgaaaatc
atcgtacaaaacattcaagatatatgatgaattatttgattattaccacg
tcagtagttatgaagataagattttacagtacacgttgatataaagatga
gatttcgctgtacacgtcagttatttagataaaatttcacagtacacgtc
agttataaagataagatttcacagtacacatcagttataaagataagatt
tcacagtacacgtcagttataaagataagatttcattgtacacgttagtt
ataaagataagatttcacaatacacgtcagccctaacacaaaacatacta
g (552 bp).
PSDC8:
(SEQ ID NO: 10)
tgattattaccacgtcagtagttatgaagataagattttacagtacacgt
tgatataaagatgagatttcgctgtacacgtcagttatttagataaaatt
tcacagtacacgtcagttataaagataagatttcacagtacacatcagtt
ataaagataagatttcacagtacacgtcagttataaagataagatttcat
tgtacacgttagttataaagataagatttcacaatacacgtcagccctaa
cacaaaacatactag (265 bp).
PTMM-AS:
(SEQ ID NO: 11)
ttcgaaattgtcagtgtgtatatgtataattctgaagaaacaattggtgg
gtccataaaaacgttcgcaatgtctttatgactttcacgggataactcga
ggagcgaaataaataaaattcatgattctcgatttctgcatttattgctt
tcacgagatattcccttcattcgtcttgtgttgggccctagatccgaccg
cttcgtgaacgggacccgacgtctccttatcgtttactttttttttattt
gggctgagcccattttcccatttacgatatgttgggctattattcaattt
gtattttgaactgggcactgtatttgtttttgtaacaaaccgtagatagc
aattgataaaaagtttcatatgtttgattctgaaccttaaaatcattatt
ttgttgtaacacgagaatctgaataaacggacgcgtggtctgtgcatacg
aaaagcacatgcccatggagcaaca (475 bp).
PTMM-IR:
(SEQ ID NO: 12)
ctgttgctccatgggcatgtgcttttcgtatgcacagaccacgcgtccgt
ttattcagattctcgtgttacaacaanataatgattttaaggttcagaat
caaacatatgaaactttttatcaattgctatctacggtttgttacaaaaa
caaatacagtgcccagttcaaaatacaaattgaataatagcccaacatat
cgtaaatgggaaaatgggctcagcccaaataaaaaaaaagtaaacgataa
ggagacgtcgggtcccgttcacgaagcggtcggatctagggcccaacaca
agacgaatgaagggaatatctcgtgaaagcaataaatgcagaaatcgaga
atcatgaattttatttatttcgctcctcgagttatcccgtgaaagtcata
aagacattgcgaacgtttttatggacccaccaattgtttcttcagaatta
tacatatacacactgacaatttcgaaactagggccatgcacaacaacacg
tactcacaaaggtgtcaatcgagcagcccaaaacattcaccaactcaacc
catcatgagcccacacatttgttgtttctaacccaacctcaaacccgtat
tctcttccgccacctcatttttgtttagttcaacacccgtcaaactgcat
cccaccccgtggactaggttcgaaattgtcagtgtgtatatgtataattc
tgaagaaacaattggtgggtccataaaaacgttcgcaatgtctttatgac
tttcacgggataactcgaggagcgaaataaataaaattcatgattctcga
tttctgcatttattgctttcacgagatattcccttcattcgtcttgtgtt
gggccctagatccgaccgcttcgtgaacgggacccgacgtctccttatcg
tttactttttttttatttgggctgagcccattttcccatttacgatatgt
tgggctattattcaatttgtattttgaactgggcactgtatttgtttttg
taacaaaccgtagatagcaattgataaaaagtttcatatgtttgattctg
aaccttaaaatcattattttgttgtaacacgagaatctgaataaacggac
gcgtggtctgtgcatacgaaaagcacatgcccatggagcaacag
(1144 bp).
PFAD2-S:
(SEQ ID NO: 13)
atcagttaacacttattaagaacaaaaatgtggtttcttgtgagaaaaat
ggtttaataaaaatccgtgattgatagaagaaaaagatcaaaataaatgg
ttggtgacgggtgatcttaaaaatgttgaaattaaggtgtgtcgtcgtta
tacgcggtaaatagatagatagaaaaatagaagtccaatgcaagagactt
aacttaatcatcccaattaattgattgcattaacttgtacttgtattttc
cgtccgccacctaatttgattaataatataataaagattacaattgaaaa
cataaacaagagaaaatccgcacgaatctaccaaagtgcatcacgtttgg
gtatccatacacgtgaccaccagtccaccacaacacaatgtctgtagata
ttttaatgtttcacatgatagaagaagccaaacgtaagaactctcttttc
cacttttagccctttccccgcctaccactgcttacgacttgtgtaagtgg
caaactagtaataatagagacgaaacttaaatataaaaaagttgaatcca
accaagttggtgttaatcaaatggttaagttataatggtgaaagatttgc
catgtgtattgtattaagagttaagaccaaggtttggttcccatcactta
cgattctttcttttcatatgattctaaagttagttattataaacatctta
atttactacacaatattcggtaatttctacatattttagagattagtttg
agtttcaatccatactttactagtgattataaattaatatacgtactttt
cgactataaagtgaaactaagtaaattagaacgtgatattaaaaagttaa
tgttcactgttatatttttttcacaagtaaaaaatgggttatttgcggta
aataaaaataccagatattttgaattgattaaaaaggttgaaataagaga
ggaggggaaagaaaagaaggtgggggcccagtatgaaagggaaaggtgtc
atcaaatcatctctctctctctctctctaccttcgacc (1038 bp).
PCH42-S:
(SEQ ID NO: 14)
tcgagactaaatttcagggacaacgagcagcacgaaactaagtttaggag
aaagtgcatcttagcattgatcgaacatttcttactcaaatctacgcata
aacgtcacctctaacacagaaatgttcatcgattatgatcaaccgatcgt
caatcgtcgaaccttagcaaaccgaagctaaaacaacgcctgacagtgag
attctactcaatcgacgagcaacgagggtaaattcttaccgattgaatcg
attgcagttgtatatgtagtaggcgctaacgataaacgttcaacggcaac
aagacgacgacaccggagagaaaatcgccgacggaactcgaaggggggag
attttgaaattagctgggctccgaatgatttgacttgggcctttatattc
ttatgtttggtatatatatatagaagctcgcctttttttttggtacggct
attttattttctatatcatttttga (476 bp).
Primers:
PSDC f1,
(SEQ ID NO: 19)
CACCTAGGAAAATGAGGAAAGTGTTGTGCCA
PSDC r2,
(SEQ ID NO: 20)
CGGCGCGCCCACCCTTAATAGTCGACTCTAGACTAGTCTAGTATGTTTTG
TGTTAGGGC
PSDC1 f1,
(SEQ ID NO: 21)
CACCTAGGAAAATGAGGAAAGTGTTGTGCCA
PSDC1 r2,
(SEQ ID NO: 22)
TACTCGAGTCTAGACTAGTAATAATTCATCATATATCTTGA
PSDC2 f1,
(SEQ ID NO: 23)
CACCTAGGAAAATGAGGAAAGTGTTGTGCCA
PSDC2 r2,
(SEQ ID NO: 24)
ACTCGAGTCTAGACTAGTATGGTGTATTGGAGATTCTA
PSDC3 f1,
(SEQ ID NO: 25)
CACCTAGGAAAATGAGGAAAGTGTTGTGCCA
PSDC3 r2,
(SEQ ID NO: 26)
TACTCGAGTCTAGACTAGTTTCGTCGGAGATCACCTTTCT
PSDC4 f1,
(SEQ ID NO: 27)
CACCTAGGAAAATGAGGAAAGTGTTGTGCCA
PSDC4 r2,
(SEQ ID NO: 28)
TACTCGAGTCTAGACTAGTGTCTTTGCAGAGACATGAAAGA
PSDC5 f1,
(SEQ ID NO: 29)
CACCTAGGAATTTTCTTACTTTTAAAGACA
PSDC5 r2,
(SEQ ID NO: 30)
CGGCGCGCCCACCCTTAATAGTCGACTCTAGACTAGTCTAGTATGTTTTG
TGTTAGGGC
PSDC6 f1,
(SEQ ID NO: 31)
CACCTAGGAAATTAATAACCTAATTTATCA
PSDC6 r2,
(SEQ ID NO: 32)
CGGCGCGCCCACCCTTAATAGTCGACTCTAGACTAGTCTAGTATGTTTTG
TGTTAGGGC
PSDC7 f1,
(SEQ ID NO: 33)
CACCTAGGGTTGGGAAACAAACCAACTTTTTACA
PSDC7 r2,
(SEQ ID NO: 34)
CGGCGCGCCCACCCTTAATAGTCGACTCTAGACTAGTCTAGTATGTTTTG
TGTTAGGGC
PSDC8 f1,
(SEQ ID NO: 35)
CACCTAGGTGATTATTACCACGTCAGTA
PSDC8 r2,
(SEQ ID NO: 36)
CGGCGCGCCCACCCTTAATAGTCGACTCTAGACTAGTCTAGTATGTTTTG
TGTTAGGGC
PTMM-S f1,
(SEQ ID NO: 37)
CACCTAGGCTGTTGCTCCATGGGCATGTGCT
PTMM-S r2,
(SEQ ID NO: 38)
CGGCGCGCCCACCCTTAATAGTCGACTCTAGACTAGTTTCGAAATTGTCA
GTGTGTA
PTMM-AS f1,
(SEQ ID NO: 39)
CACCTAGGTTCGAAATTGTCAGTGTGTA
PTMM-AS r2,
(SEQ ID NO: 40)
CGGCGCGCCCACCCTTAATAGTCGACTCTAGACTAGTCTGTTGCTCCATG
GGCATGTGC
PFAD2-S f1,
(SEQ ID NO: 41)
CACCTAGGATCAGTTAACACTTATTAAGA
PFAD2-S r2,
(SEQ ID NO: 42)
TCGGCGCGCCCACCCTTAGTCGACTCTAGAGGTCGAAGGTAGAGAGAGAG
AGA
PCH42-S f1,
(SEQ ID NO: 43)
CACCTAGGTCGAGACTAAATTTCAGGGA
PCH42-S r2,
(SEQ ID NO: 44)
ACTCGAGTCGACTCTAGACTAGTTCAAAAATGATATAGAAAATAAAATA

### Example 2

### Plant Materials and Transformation

Arabidopsis thaliana WT (Col-0) and tobacco Nicotiana benthamiana were used in this research. Stable transformation of Arabidopsis was performed by floral dip method as described by Zhang et al. (2006). Transformed plants were screened on selection medium (1×MS salts+1% Sucrose+0.5 g/L MES+100 mg/L Carbenicilin+10 mg/L basta+8% Agar pH 5.7). Two-week old T1 and T2 seedlings were used to score for clustered stomata phenotype under a microscope. T3 homozygous seedlings were used for DNA methylation and chromatin modification assays. Tobacco N. benthamiana leaf Agro-infiltration (Voinnet et al., 2003) was used to assay small RNAs levels produced by different constructs.

### Example 3

### Stomata Phenotype Observation

Cotyledons of two-weed old T1 and T2 seedlings were used to score for stomata phenotype under a microscope. In wild type (WT), stomata appear on the lower surface of cotyledons and they are separated by at least one intervening epidermal cell. If two or more than two stomata are located together, the seedling will be scored as displaying a tmm mutant phenotype (clustered stomata phenotype). For each transformation event of a construct, 32-64 T1 independent lines and 24 T2 independent lines were scored to determine the penetration rate of the tmm phenotype.

### Example 4

### DNA Methylation Analysis

Arabidopsis genomic DNA was extracted from 2-week-old seedlings by DNeasy Plant Mini Kit (Qiagen). Bisulfite DNA conversion was performed using 1 μg genomic DNA and EpiTech Bisulfite Kit (Qiagen) following the manufacturer's protocol. PCR was performed using primers located outside of the targeted region and designed for single strand methylation detection. PCR products were then cloned into pCR2.1 using TA cloning kit (Invitrogen). For each genotype, at least 20 independent clones were sequenced using Ml 3R primer. Data were analyzed by Cymate (Hetzl et al., 2007).

### Example 5

### Chromatin Precipitation (ChIP)

Three grams of 2-week-old seedlings were used in immunoprecipitation experiments as described by Gendrel et al. (Gendrel et al., 2005) with minor modifications. Cross-linked chromatin pellets resuspended in nuclei lysis buffer were sonicated in a Bioruptor (Bioruptor UCD 200, Diagenode) for 10 min at the maximum level. Samples were sonicated for periods of 30 sec. with 30 sec. interval in between treatments. Histone H3 trimethyl Lys4 (K4me3) antibody were from Active Motif (Cat. No. 39159). Histone H3 Acetylation, Histone H3 trimethyl Lys9 (K9me3), Histone H3 trimethyl Lys27 (K27me3) antibodies were from Milipore (Cat. No. 06-599, 07-442, 07-449).

### Example 6

### Small RNA Analysis

Total RNA was extracted from 2-week-old seedlings by Trizol Reagent (Invitrogen) following the manufacturer's instructions. Small RNA libraries were constructed by TruSeq Small RNA Sample Prep Kit (Illumina) following the manufacturer's instructions. Briefly, 1 μg total RNA or purified small RNA was ligated with 3′ and 5′ adaptors and used as a template for RT-PCR. After PCR amplification, 6 μl of each sample were pooled and separated on a 6% polyacrylamide Gel. Gel slices corresponding to ˜20-30nt small RNAs were recovered. Sequences were determined by Illunina HiSeq in the Genomic Center of Rockefeller University. Adapter sequence was trimmed by local Perl script and only reads longer than 15nt were used in further analysis. All retained reads were mapped to the Arabidopsis genome (TAIR 9 version) by C program allowing no mismatch.

### Example 7

### Small RNA Northern Blots

RNA gel blots were analyzed using 10 μg of total RNA per lane. RNA was separated by 17% PAGE/8 M Urea/0.5×TBE buffer (National Diagnostics, USA). The gel was electroblotted to Hybond N+ membrane (Amersham, Piscataway, N.J.) and then UV cross-linked. The probes were made by in vitro transcription of DNA fragments from the candidate promoter region. Hybridizations were performed at 42° C. overnight in UltraHyb hybridization solution (Ambion, Austin, Tex.), according to the supplier's direction. After hybridization, membranes were washed in 2×SSC with 0.1% SDS and analyzed using BioMax MS films (Kodak).

### Example 8

### SDC Promoter Fragment (-274 to −2,004) Enhance Transcriptional Gene Silencing (TGS) of TMM when Co-Expressed with a TMM Promoter DNA Fragment

Too Many Mouths (TMM) encodes a leucine-rich repeat receptor-like protein that is expressed in proliferative post protodermal cells. It is able to sense positional cues and control the minimal one-celled stomata spacing pattern. Disruption of TMM will cause formation of stomata clusters (tmm mutant phenotype) which can be clearly seen in cotyledons (Yang et al., 1995). Over-expressing a hairpin structure of TMM promoter sequence (-9 to −483) was able to silence TMM efficiently showing that this locus is a good candidate for transcriptional gene silencing (TGS) research. When over-expressing a sense strand of TMM promoter (-9 to −483), only a weak tmm mutant phenotype was observed with a low penetration rate. We used this weak TGS system to screen for sequences that can enhance TGS.

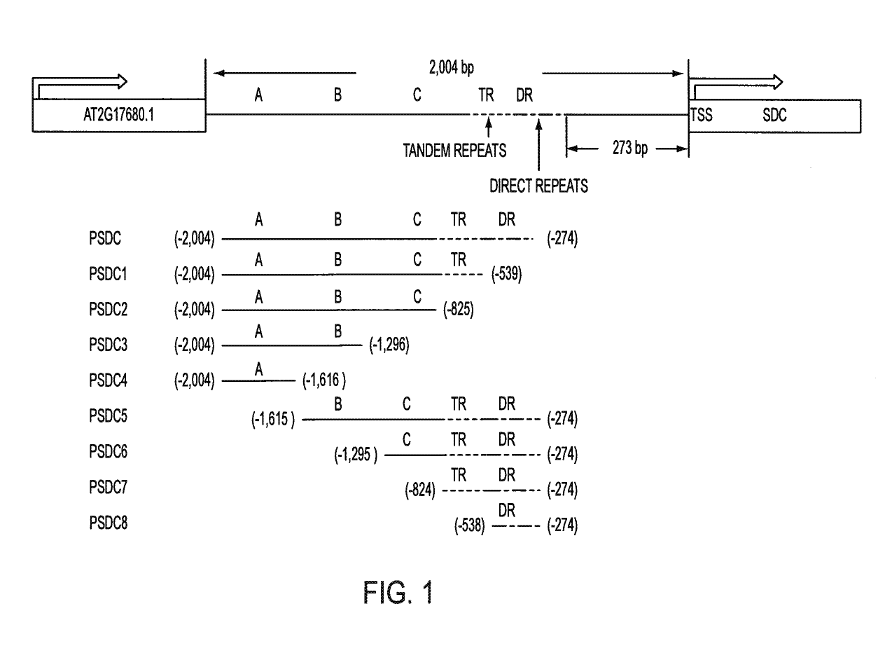

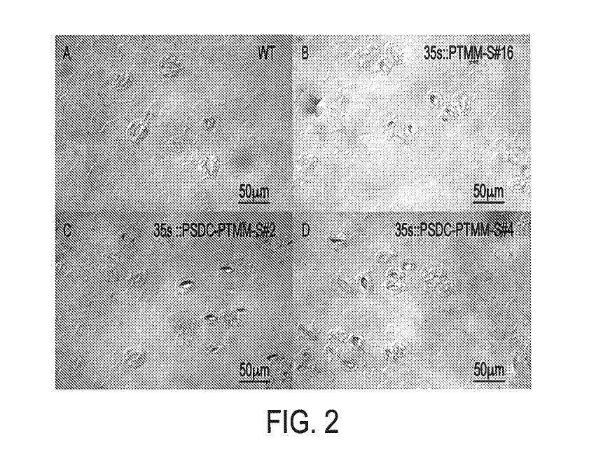

In the Arabidopsis genome, many DNA loci are silenced by higher levels of endogenous 24nt siRNAs and these loci are heavily methylated. One of these loci is the promoter of SUPPRESSOR OF ddc (SDC) which encodes an F-box protein of unknown function. SDC is silenced in WT and reactivated by more than 250 fold in ddc mutants in which asymmetric DNA methylation was abolished (Henderson et al., 2008). To test whether this locus contains sequences that can promote TGS, we cloned a 1,731-bp DNA fragment from the promoter region (-274 to −2,004; transcriptional start site is +1, FIG. 1) of SDC and fused to the 5′ end of TMM promoter DNA fragment (-9 to −483; transcription start site is +1). Four constructs, pBA/PTMM-S, pBA/PSDC-PTMM-S, pKGWFS7/PTMM-S, and pKGWFS7/PSDC-PTMM-S were transformed into Arabidopsis. Note that pBA/PTMM-S and pBA/PSDC-PTMM-S contain a 35s promoter whereas pKGWFS7/PTMM-S and pKGWFS7/PSDC-PTMM-S do not have any promoters. Transgenic plants were scored for clustered stomata phenotype (tmm) under a microscope. In WT, stomata are separated by intervening epidermal cells (FIG. 2a). In PTMM-S plants, 3 or 4 stomata were located in clusters (FIG. 2b). In PSDC-PTMM-S plants, the clustered stomata phenotype was more severe than PTMM-S plants (FIG. 2c, 2d). Phenotype penetration rates were scored (Table 1) using two-week old seedlings. In T1 generation, none of the PTMM-S plants showed any tmm phenotype whereas transgenic plants of PSDC-PTMM-S showed tmm mutant phenotype at penetration rate of 18.8%. In T2 generation, 4-8 seedlings grown on selection medium were examined for each line and a total of 24 lines were analyzed for each construct. Transgenic seedlings with PTMM-S showed a penetration rate of 12.5%, which was increased to 50.5% in PSDC-PTMM-S plants. No clustered stomata phenotype was observed in either T1 or T2 generation in pKGWFS7/PTMM-S and pKGWFS7/PSDC-PTMM-S plants where no promoter was placed upstream of these DNA sequences. These results indicate that PSDC is able to promote clustered stomata phenotype when co-expressed with the TMM promoter DNA fragment.

TABLE 1
Phenotype Penetration Rates

| | T1 Lines | | | T2 Lines | | |
|---|---|---|---|---|---|---|
| | Number of | | | Number of | | |
| | plants with | Total | Penetration | plants with | Total | Penetration |
| Construct | phenotype | Number | rate (%) | phenotype | Number | rate (%) |
| 35S::PTMM-S | 0 | 32 | 0 | 3 | 24 | 12.5 |
| 35S::PSDC-PTMM-S | 6 | 32 | 18.8 | 12 | 24 | 50.5 |
| No promoter::PTMM-S | 0 | 32 | 0 | 0 | 24 | 0 |
| No promoter::PSDC-PTMM-S | 0 | 32 | 0 | 0 | 24 | 0 |

### Example 9

### SDC Promoter Enhances Clustered Stomata Phenotype More Efficiently without its Direct Repeats

The SDC promoter contains seven direct repeats (DR) located at −296 to −518 upstream of the SDC transcriptional start site. This DR region contains higher levels of related 24nts siRNAs which presumably mediate heavy DNA methylation (Henderson et al., 2008). Thus, the DR sequences may be responsible for the enhancement of TGS of TMM locus. To test this hypothesis, the SDC promoter DR region (PSDC8, FIG. 1) and SDC promoter sequence without DR (PSDC1, FIG. 1) were cloned and separately fused to PTMM-S to give two new constructs, pBA/PSDC8-PTMM-S and pBA/PSDC1-PTMM-S. These two constructs were transformed into Arabidopsis to generate stable transgenic plants. Clustered stomata phenotype was scored at T1 and T2 generation. Table 2 shows that in T1 generation, none of the 50 independent lines carrying PSDC8-PTMM-S showed a clustered stomata phenotype. By contrast, both PSDC-PTMM-S and PSDC1-PTMM-S plants produced a penetration rate of 18.8% and 50% of tmm mutant phenotype, respectively. In T2 generation, PSDC1-PTMM-S plants showed a higher penetration rate (87.5%) compared to PSDC-PTMM-S plants (50.5%). The DR region appeared to play a negative role in TGS as the penetration rate of PSDC8-PTMM-S plants (4.2%) was even lower than PTMM-S plants (12.5%). These results indicate that both SDC promoter sequence with or without DR are able to increase penetration rates of tmm mutant phenotype by enhancing TGS. Moreover the SDC promoter sequence without DR is more efficient and the DR region has a suppressive effect.

TABLE 2
Phenotype Penetration Rates

| | T1 Lines | | | T2 Lines | | |
|---|---|---|---|---|---|---|
| | Number of | | | Number of | | |
| | plants with | Total | Penetration | plants with | Total | Penetration |
| Construct | phenotype | Number | rate (%) | phenotype | Number | rate (%) |
| 35S::PTMM-S | 0 | 32 | 0 | 3 | 24 | 12.5 |
| 35S::PSDC-PTMM-S | 6 | 32 | 18.8 | 12 | 24 | 50.5 |
| 35S::PSDC1-PTMM-S | 24 | 48 | 50 | 21 | 24 | 87.50 |
| 35S::PSDC8-PTMM-S | 0 | 50 | 0 | 1 | 24 | 4.2 |

### Example 10

### Stomata Clustering Phenotype is Caused by TGS of the TMM Locus

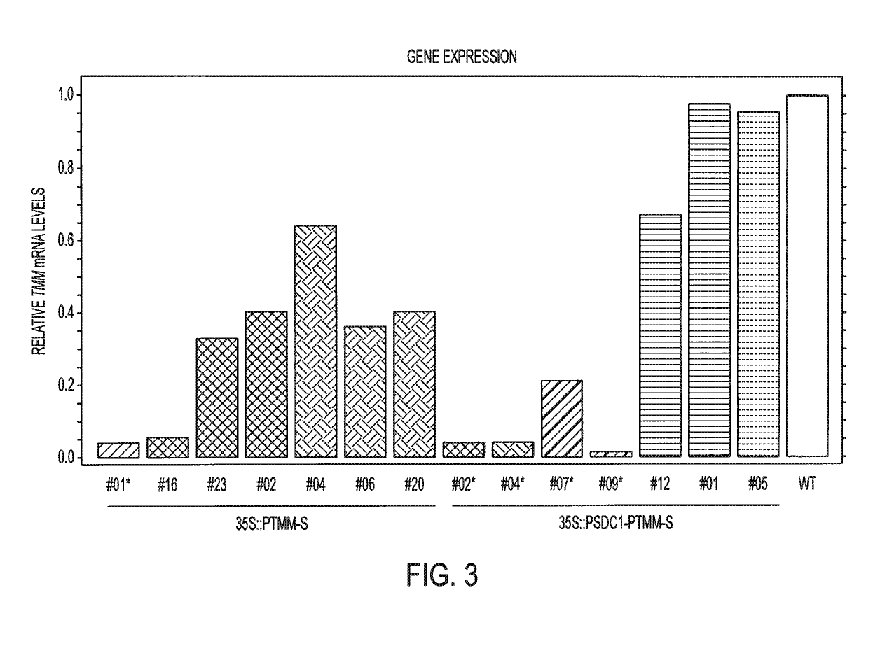

To examine the relationship of tmm mutant clustered stomata phenotype and TMM mRNA level, RNAs were prepared from two-week old seedlings of several T2 lines of both PTMM-S and PSDC1-PTMM-S plants with or without phenotype. TMM mRNA levels in total RNA were checked by real-time PCR. FIG. 3 shows that in PTMM-S plants, lines with tmm phenotype (line #1, #16) expressed much lower TMM mRNA levels compared to those lines without any tmm phenotype (line #23, #2, #4, #6, and #20). Similar results were obtained with PSDC1-PTMM-S plants. These results show that the clustered stomata phenotype is related to lower TMM mRNA expression levels.

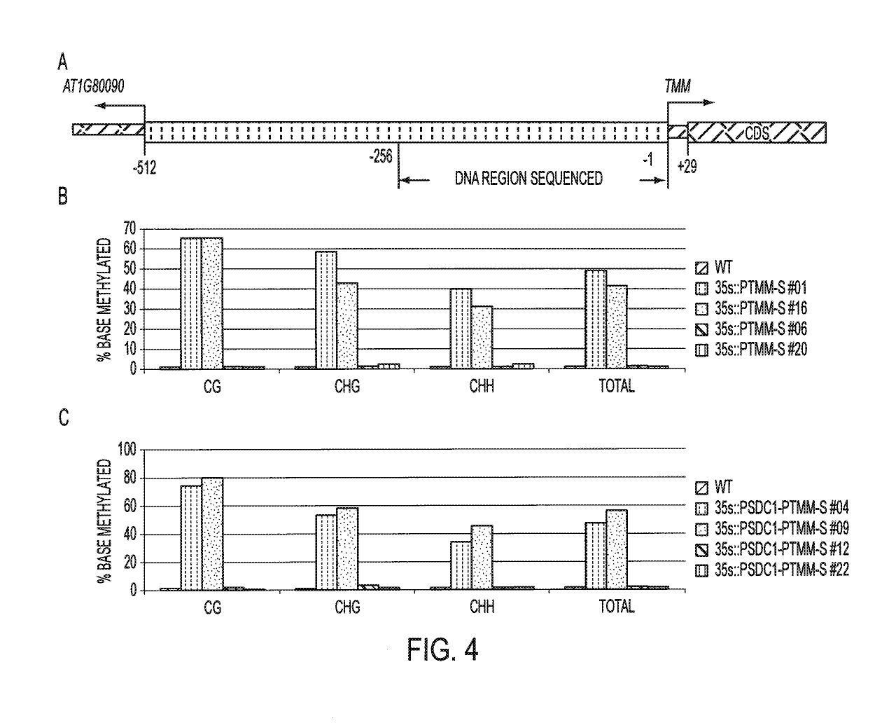

Increased DNA methylation at promoter region is generally considered as the hallmark for TGS (Aufsatz et al., 2002). To determine whether the clustered stomata is related to TMM promoter DNA methylation, we analyzed T3 lines of PTMM-S and PSDC1-PTMM-S plants. Transgenic plants without tmm phenotype were used as controls. All CG, CHG (H means A, C, or T), and CHH DNA methylation were significantly increased at the TMM promoter region in all four tested lines with phenotype (PTMM-S line #1, #16 and PSDC1-PTMM-S line #4, #9). By contract, transgenic lines (PTMM-S line #6, #20 and PSDC1-PTMM-S line #12, #22) that did not display any phenotype showed WT DNA methylation levels (FIGS. 4b and 4c).

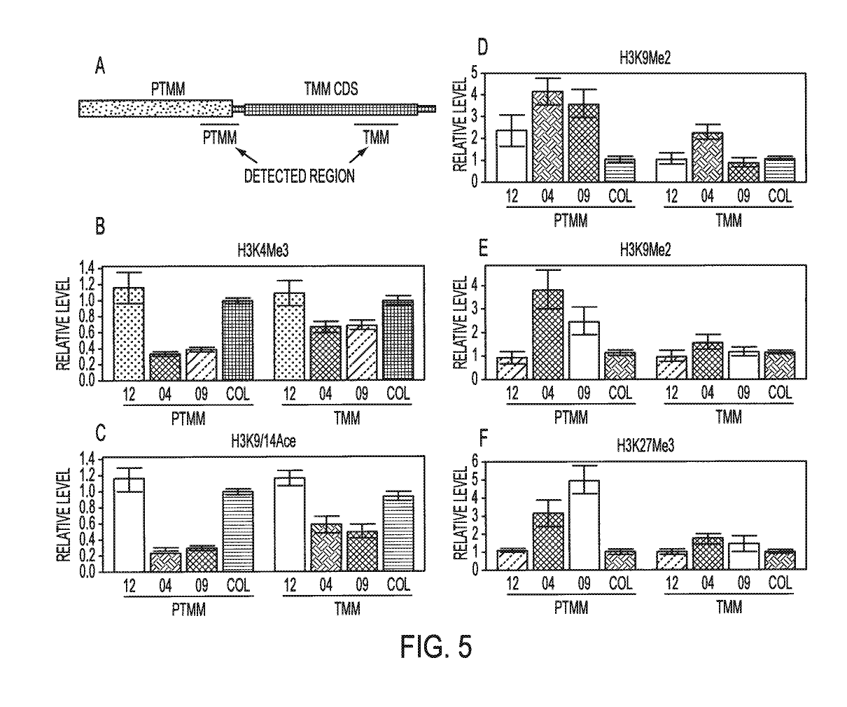

To determine whether the changes in DNA methylation observed at the TMM promoter region were accompanied by changes in histone modifications, we used chromatin immunoprecipitation (ChIP) assay to investigate H3 acetylation and H3K4 trimethylation for active markers and H3K9me2, H3K9me3 and H3K27me3 for repressive markers. Two primer pairs were designed to interrogate promoter and coding region of the endogenous TMM locus (FIG. 5a). Significant reduction of active markers, H3K9/14Ac and H3K4me3 was observed in two positive lines (PSDC1-PTMM-S line #4, #9) at the TMM promoter regions, compared to negative lines (PSDC1-PTMM-S line #12) and WT plants (FIGS. 5b and 5c). For chromatin repressive markers, positive transgenic plant lines showed higher levels at the TMM promoter, than negative transgenic plant lines and WT. (FIGS. 5d-5f). In TMM coding region, there were no clear difference among all tested lines and WT for any chromatin modification (FIGS. 5b-5f).

Positive transgenic plants with lower TMM mRNA levels, higher DNA methylation, lower levels active chromatin modification markers, and higher levels of repression chromatin markers confirmed that the tmm phenotype in transgenic plants is a consequence of TGS of the TMM locus.

### Example 11

### SDC Promoter Sequences Promote siRNA Production

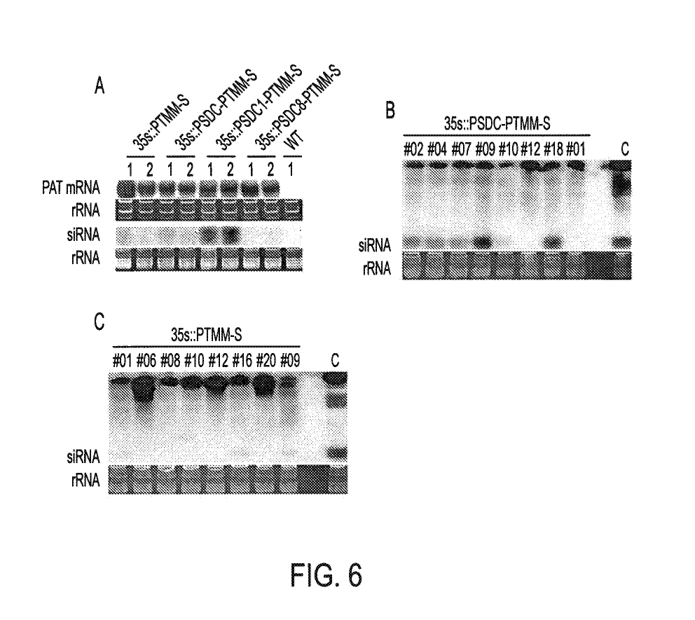

Small RNAs especially 24nt siRNAs are incorporated into Ago4 complex which guides a de novo DNA methylation of targeted genomic locus. An abundance of siRNA is associated with the upstream region of SDC promoter suggesting it may contain certain features favorable for siRNAs production. The ability to increase siRNA production may explain its function as an enhancer of TGS. To test this hypothesis, Agrobacteria carrying constructs of pBA/PTMM-S, pBA/PSDC-PTMM-S, pBA/PSDC1-PTMM-S, or PBA/PSDC8-PTMM were infiltrated into tobacco leaves. Two leaves were infiltrated with each construct and mRNA of PAT (phosphinothricin acetyl transferase) was used as an internal control to assess transformation efficiencies. Total RNAs were extracted from these leaves two days after infiltration. Northern blots analysis (FIG. 6a) showed that PTMM-related siRNA was expressed at a high level in leaves infiltrated with PSDC1-PTMM-S but at a lower level in leaves infiltrated with PTMM-S. In PSDC8-PTMM-S leaves, there were no or very lower levels of PTMM-related siRNA expressed. These results show that the SDC promoter without direct repeats (PSDC1) can enhance PTMM-related siRNA production but the direct repeat (PSDC8) plays a repressive role. Comparing PTMM-related siRNA levels and penetration rates of clustered stomata phenotype (FIGS. 2a-2d), we found a direct correlation between the two. Thus, promoting siRNA production is at least one important reason for the SDC promoter sequence functioning as an enhancer of TGS at the TMM locus. Some T2 Arabidopsis stable transgenic lines expressing either PTMMS or PSDC1-PTMM-S were also analyzed by northern blots (FIGS. 6b and 6c). The results confirmed that PSDC1-PTMM plants expressed a high level of PTMM-related siRNA compared to PTMM-S transgenic plants.

### Example 12

### Majority of PTMM-Related siRNAs were 24Nts

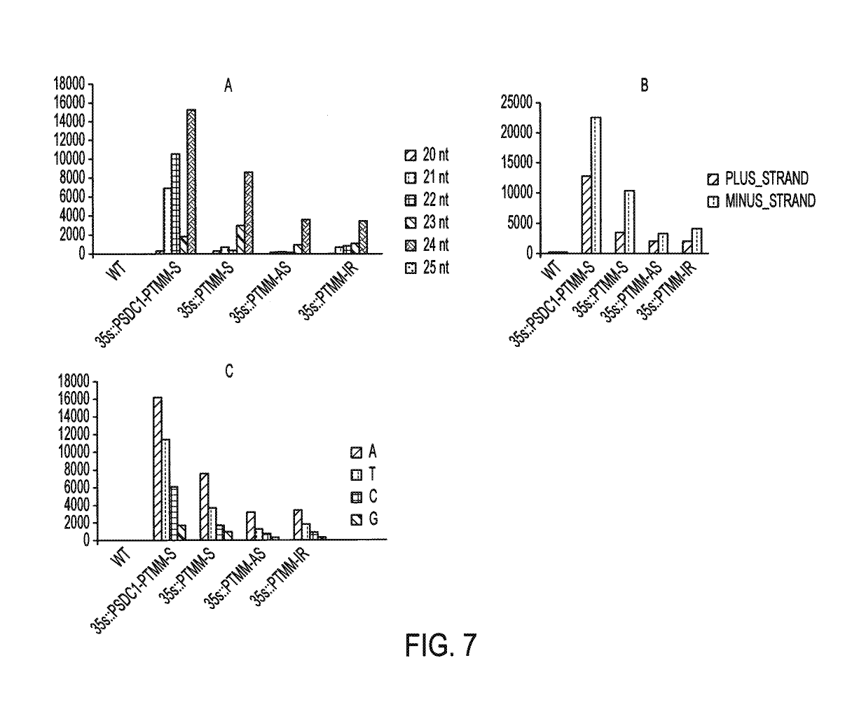

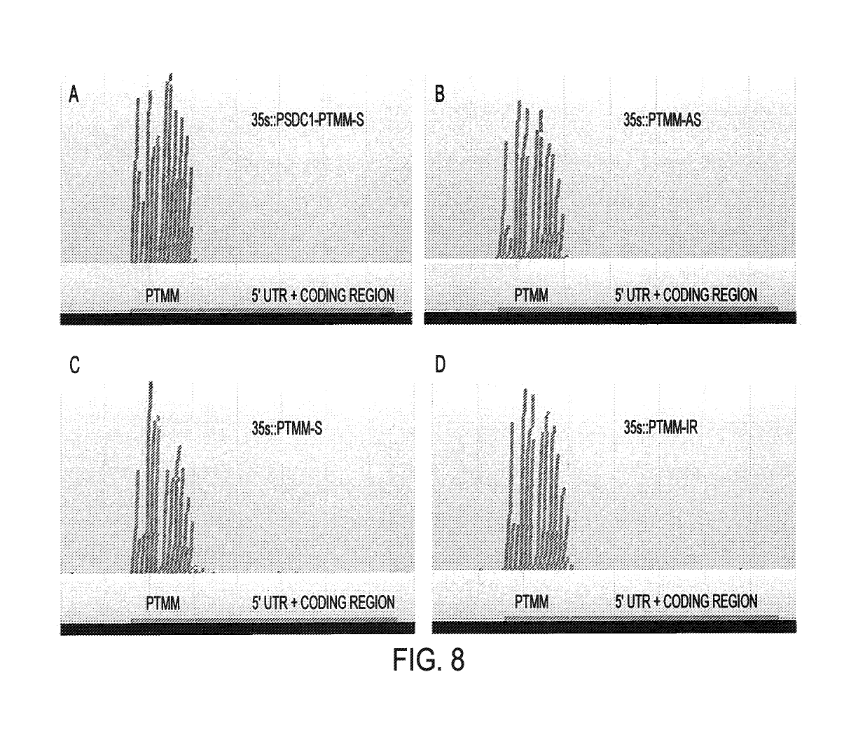

There is good evidence that TGS can be induced through RdDM with smRNAs, mostly 24-26nt, derived from exo/endo-geneous double-stranded inverted repeat (IR) RNAs (Wassenegger et al., 1994; Mette et al., 2000; Hamilton et al., 2002). On the other hand, shorter smRNA species (21-22 nt) mainly mediate degradation of target RNAs with sequence homology resulting in post-transcriptional gene silencing (PTGS) (Hamilton et al., 2002; Vaucheret, 2006). To investigate small RNA features of positive transgenic plants, small RNAs from PTMM-S, PSDC1-PTMM, PTMM-AS, and PTMM-IR positive lines were sequenced and total reads were summarized in Table 3. We obtained about 30 million total reads for each sample and small RNAs with length of 20 to 25 were analyzed in detail. The majority of small RNAs were 24nt in length (FIG. 7a) indicating a correlation of PTMM-related siRNAs with TGS of TMM locus. The preference for the first base of the small RNA was A (FIG. 7c) favoring their incorporation into Ago4 complex (Mi et al, 2008). We also found that small RNAs were mainly derived from the minus strand (FIG. 7b). Small Analysis of RNAs distribution (FIGS. 8a-8d) on the TMM locus showed that there were no small RNAs corresponding to the TMM coding region confirming that the tmm mutant phenotype was caused by TGS.

TABLE 3

| | | | Unique Reads |
|---|---|---|---|
| | Total Reads | Unique Reads | (19-28 nt) |
| WT | 29,942,636 | 8,656,293 | 5,723,487 |
| 35S::PSDC1-PTMM-S | 33,447,020 | 10,168,998 | 5,937,487 |
| 35S::PTMM-S | 26,970,285 | 7,982,744 | 5,125,099 |
| 35S::PTMM-AS | 29,520,225 | 8,749,079 | 4,824,961 |
| 35S::PTMM-IR | 30,510,016 | 9,204,333 | 5,126,612 |

### Example 13

### SDC Promoter 5′ Region is Critical for Promoting siRNA Production

The observation that the SDC promoter without direct repeats (DR) can enhance PTMM-related siRNAs production suggests the presence of a siRNA production enhancer upstream of the DR sequence. To investigate this DNA sequence, the SDC promoter was divided into five regions according to small RNA and DNA methylation information from the Anno-J: Arabidopsis epigenome maps website (FIGS. 8a-8d). The DR sequences produce abundant small RNAs associated with severe DNA methylation in WT which is abolished in ddc mutant. A tandem repeat (TR) located upsteam of DR produces similar levels of small RNAs and DNA methylation between WT and mutants. Regions B and C contain DNA methylation peaks in WT which are lost in met1 mutant. In region A, the small RNA level is increased dramatically in rdd mutant compared to in WT. Truncated PSDC fragments, designed by removing serial sequences from 5′ or 3′ end of the SDC promoter (FIG. 1), were cloned and fused to the 5′end of PTMM-S to generate the follow constructs: pBA/PSDC-PTMM-S, pBA/PSDC1-PTMM-S, pBA/PSDC2-PTMM-S, pBA/PSDC3-PTMM-S, pBA/PSDC4-PTMM-S, pBA/PSDC5-PTMM-S, pBA/PSDC6-PTMM-S, pBA/PSDC7-PTMM-S, and pBA/PSDC8-PTMM-S.

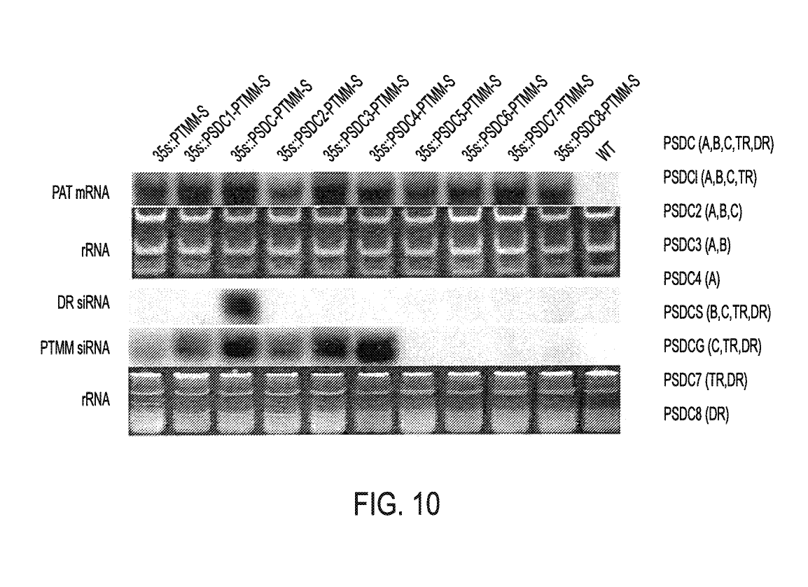

PTMM-related siRNAs were analyzed by northern blots using total RNAs extracted from tobacco leaves infiltrated by Agrabacteria carrying these constructs. The level of mRNA of PAT, a selection marker of these constructs, was used as an inner control to monitor tobacco leaf transformation efficiency. FIG. 10 shows that these PSDC truncated constructs can be divided into two groups according to PTMM-related siRNAs levels. PTMM-related siRNAs were expressed higher than PTMM-S control (FIG. 10, the first lane from left) in the first group (FIG. 10, lane 2 to 6 from left) but lower in the second group (FIG. 10, lane 7 to 10 from left). These results indicate that the siRNA production enhancer is in the first but not in the second group. According to the truncated SDC promoter information (FIG. 1), all first group constructs contain SDC promoter 5′region sequence (region A) which is absent from the second group. Thus, the region A promotes siRNAs production. To further confirm this point, we also analyzed DR-related siRNA production (FIG. 10). There were five constructs (pBA/PSDC-PTMM-S, pBA/PSDC5-PTMM-S, pBA/PSDC6-PTMMS, pBAPSDC7-PTMMS, and pBA/PSDC8-PTMM-S) containing DR sequence. Only the construct (pBA/PSDC-PTMM-S) with region A expressed high level of DR-related siRNAs. All the others (without region A) did not produce or produced very lower levels of DR-related siRNAs.

### Example 14

### SDC Promoter Region a is Able to Promote siRNAs Production of Other DNA Fragments

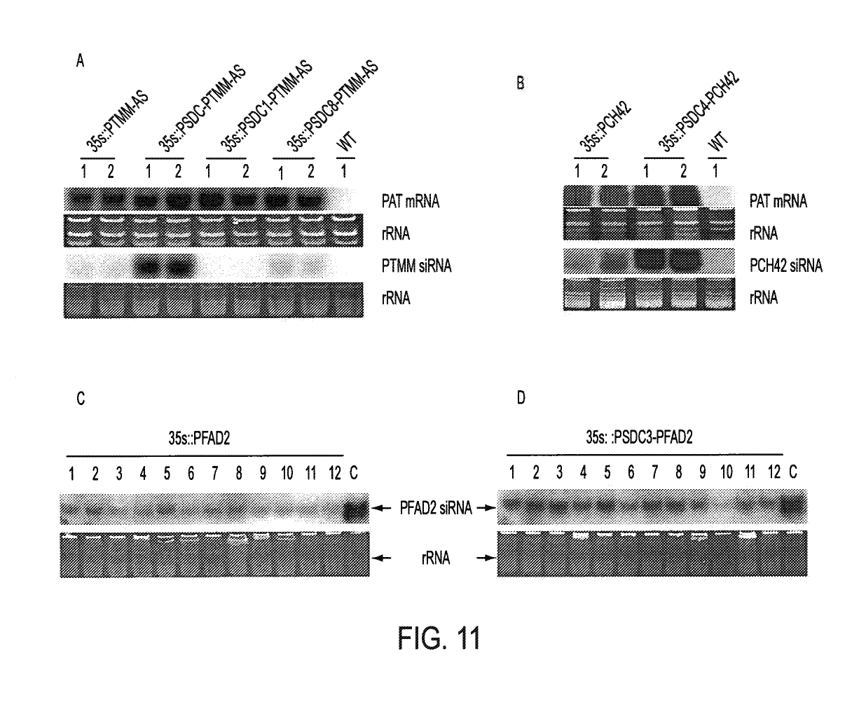

The region A sequence of the SDC promoter can enhance PTMM-related siRNA production. We wanted to know whether this promoting function also apply to other loci. To address this issue, TMM promoter anti-sense DNA fragment (-9 to-483; transcriptional start site is +1) was cloned and fused to the 3′end of different truncated PSDC sequences to generate pBA/PTMM-AS, pBA/PSDC-PTMM-AS, pBA/PSDC3-PTMM-AS, and pBA/PSDC7-PTMM-AS. All these constructs were transformed into tobacco leaves for northern blot analysis. In this experiment, two leaves were infiltrated for each construct and mRNA levels of PAT were used as an internal control to assess transformation efficiency. PTMM-AS related siRNAs were expressed at higher levels in PSDC3-PTMM-AS (with region A) leaves than in PTMM-AS leaves This result shows that region A is able to enhance production of siRNAs related to TMM promoter anti-sense DNA fragment (FIG. 11a). The result also shows that DR without region A (PSDC7) displayed a negative effect on PTMM-AS related siRNA production. Similar experiment was performed in CH42 promoter DNA fragment (-40 to −515; transcriptional start site is +1). PCH42 related siRNAs were expressed at a high levels in PSDC4-PCH42 (with region A) leaves but not in PCH42 leaves (FIG. 11b). FAD2 promoter sequence (-28 to-1,065; transcriptional start site is +1) was cloned and fused to 3′end of PSDC3 (region A) to give pBA/PFAD2 and pBA/PSDC3-PFAD2 constructs. These two constructs were transformed to Arabidopsis to generate stable transgenic lines. Two-week old seedlings were used for northern analysis. Twelve lines were selected randomly for each construct. Northern blot results (FIGS. 11c and 11d) indicated that PFAD2-related siRNAs were also higher in pBA/PSDC3-PFAD2 transgenic lines.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

Aufsatz, W., Mette, M. F., Van Der Winden, J., Matzke, A. J. M., and Matzke, M. (2002). RNA-directed DNA methylation in Arabidopsis. Proceedings of the National Academy of Sciences of the United States of America 99, 16499-16506.
Chiu, W. et al. (1996). Engineered GFP as a vital reporter in plants. Current Biology 6:325-330.
Christensen, A. H. and Quail, P. H, (1989). Sequence analysis and transcriptional regulation by heat shock of polyubiquitin transcripts from maize. Plant Mol Biol 12:619-632.
Christensen, A. H. et al. (1992). Maize polyubiquitin genes: structure, thermal perturbation of expression and transcript splicing, and promoter activity following transfer to protoplasts by electroporation. Plant Mol Biol 18:675-689.
Cigan, A. M., Unger—Wallace, E., and Haug—Collet, K. (2005). Transcriptional gene silencing as a tool for uncovering gene function in maize. The Plant Journal 43, 929-940.
Daxinger, L., Kanno, T., Bucher, E., Van Der Winden, J., Naumann, U., Matzke, A. J. M., and Matzke, M. (2009). A stepwise pathway for biogenesis of 24-nt secondary siRNAs and spreading of DNA methylation. The EMBO Journal 28, 48-57.
De Wet, J. R. et al. (1987). Firefly luciferase gene: structure and expression in mammalian cells. Mol Cell Biol 7:725-737.
Eamens, A., Wang, M. B., Smith, N. A., and Waterhouse, P. M. (2008). RNA silencing in plants: yesterday, today, and tomorrow. Plant Physiology 147, 456-468.
Garcia-Ruiz, H., Takeda, A., Chapman, E. J., Sullivan, C. M., Fahlgren, N., Brempelis, K. J., and Carrington, J. C. (2010). Arabidopsis RNA-dependent RNA polymerases and dicer-like proteins in antiviral defense and small interfering RNA biogenesis during Turnip Mosaic Virus infection. The Plant Cell 22, 481-496.
Goff, S. A. et al. (1990). Transactivation of anthocyanin biosynthetic genes following transfer of B regulatory genes into maize tissues. EMBO J 9:2517-2522.
Gendrel, A. V., Lippman, Z., Martienssen, R., and Colot, V. (2005). Profiling histone modification patterns in plants using genomic tiling microarrays. Nature methods 2, 213-218.
Haag, J. R., and Pikaard, C. S. (2011). Multisubunit RNA polymerases IV and V: purveyors of non-coding RNA for plant gene silencing. Nature Reviews Molecular Cell Biology 12, 483-492.
Hamilton, A., Voinnet, O., Chappell, L., and Baulcombe, D. (2002). Two classes of short interfering RNA in RNA silencing. The EMBO Journal 21, 4671-4679.
Havecker, E. R., Wallbridge, L. M., Hardcastle, T. J., Bush, M. S., Kelly, K. A., Dunn, R. M., Schwach, F., Doonan, J. H., and Baulcombe, D. C. (2010). The Arabidopsis RNA-directed DNA methylation argonautes functionally diverge based on their expression and interaction with target loci. The Plant Cell 22, 321-334.
Heilersig, B. H. J. B., Loonen, A. E. H. M., Janssen, E. M., Wolters, A. M. A., and Visser, R. G. F. (2006). Efficiency of transcriptional gene silencing of GBSSI in potato depends on the promoter region that is used in an inverted repeat. Molecular Genetics and Genomics 275, 437-449.
Hetzl, J., Foerster, A. M., Raidl, G., and Scheid, O. M. (2007). CyMATE: a new tool for methylation analysis of plant genomic DNA after bisulphite sequencing. The Plant Journal 51, 526-536.
Henderson, I. R., and Jacobsen, S. E. (2008). Tandem repeats upstream of the Arabidopsis endogene SDC recruit non-CG DNA methylation and initiate siRNA spreading. Genes & Development 22, 1597.
Henderson, I. R., Zhang, X., Lu, C., Johnson, L., Meyers, B. C., Green, P. J., and Jacobsen, S. E. (2006). Dissecting Arabidopsis thaliana DICER function in small RNA processing, gene silencing and DNA methylation patterning. Nature Genetics 38, 721-725.
Jefferson, R. A. et al. (1991). Plant Molecular Biology Manual, ed. Gelvin et al., Kluwer Academic Publishers, pp. 1-33.
Jones, L., Ratcliff, F., and Baulcombe, D. C. (2001). RNA-directed transcriptional gene silencing in plants can be inherited independently of the RNA trigger and requires Met1 for maintenance. Current Biology 11, 747-757.
Kain, S. R. et al. (1995). Green fluorescent protein as a reporter of gene expression and protein localization. BioTechniques 19, 650-655.
Kanno, T., Mette, M. F., Kreil, D. P., Aufsatz, W., Matzke, M., and Matzke, A. J. M. (2004). Involvement of putative SNF2 chromatin remodeling protein DRD1 in RNA-directed DNA methylation. Current Biology 14, 801-805.
Kanno, T., Huettel, B., Mette, M. F., Aufsatz, W., Jaligot, E., Daxinger, L., Kreil, D. P., Matzke, M., and Matzke, A. J. M. (2005). Atypical RNA polymerase subunits required for RNA-directed DNA methylation. Nature Genetics 37, 761-765.
Kost, B., Spielhofer, P., and Chua, N. H. (1998). A GFP—mouse talin fusion protein labels plant actin filamentsin vivoand visualizes the actin cytoskeleton in growing pollen tubes. The Plant Journal 16, 393-401.
Last, D. I. et al. (1991). pEmu: an improved promoter for gene expression in cereal cells. Theor Appl Genet 81:581-588.
Law, J. A., and Jacobsen, S. E. (2010). Establishing, maintaining and modifying DNA methylation patterns in plants and animals. Nature Reviews Genetics 11, 204-220.
Mallory, A., and Vaucheret, H. (2010). Form, function, and regulation of ARGONAUTE proteins. The Plant Cell 22, 3879-3889.
Matzke, M., Primig, M., Trnovsky, J., and Matzke, A. (1989). Reversible methylation and inactivation of marker genes in sequentially transformed tobacco plants. The EMBO Journal 8, 643-649.
Matzke, M., Kanno, T., Daxinger, L., Huettel, B., and Matzke, A. J. M. (2009). RNA-mediated chromatin-based silencing in plants. Current Opinion in Cell Biology 21, 367-376.
Matzke, M. A., and Matzke, A. J. M. (2004). Planting the seeds of a new paradigm. PLoS Biology 2, e133.
McElroy, D. et al. (1990). Isolation of an efficient actin promoter for use in rice transformation. Plant Cell 2:163-171.
Mette, M., Aufsatz, W., Van der Winden, J., Matzke, M., and Matzke, A. (2000). Transcriptional silencing and promoter methylation triggered by double-stranded RNA. The EMBO Journal 19, 5194-5201.
Mi S., Cai T., Hu Y., Chen Y., Hodges E., Ni F., Wu., L., Li S., Zhou H., Long C., Shen S., Hannon G. J., and Qi Y. (2008). Sorting of small RNAs int Arabidopsis Argonaute Complex is Directed by the 5′ Terminal Nucleotide. Cell 133, 116-127.
Miura, A., Nakamura, M., Inagaki, S., Kobayashi, A., Saze, H., and Kakutani, T. (2009). An Arabidopsis jmjC domain protein protects transcribed genes from DNA methylation at CHG sites. The EMBO Journal 28, 1078-1086.
Mlotshwa, S., Pruss, G. J., Gao, Z., Mgutshini, N. L., Li, J., Chen, X., Bowman, L. H., and Vance, V. (2010). Transcriptional silencing induced by Arabidopsis T—DNA mutants is associated with 35S promoter siRNAs and requires genes involved in siRNA—mediated chromatin silencing. The Plant Journal 64, 699-704.
Odell, J. T. et al. (1985). Identification of DNA sequences required for activity of the cauliflower mosaic virus 35S promoter. Nature 313:810-812.
Okano, Y., Mild, D., and Shimamoto, K. (2008). Small interfering RNA (siRNA) targeting of endogenous promoters induces DNA methylation, but not necessarily gene silencing, in rice. The Plant Journal 53, 65-77.
Sijen, T., Vijn, I., Rebocho, A., van Blokland, R., Roelofs, D., Mol, J. N. M., and Kooter, J. M. (2001). Transcriptional and posttranscriptional gene silencing are mechanistically related. Current Biology 11, 436-440.
Vaucheret, H. (2006). Post-transcriptional small RNA pathways in plants: mechanisms and regulations. Genes & Development 20, 759-771.
Velten, J. et al. (1984). Isolation of a dual plant promoter fragment from the Ti plasmid of Agrobacterium tumefaciens. EMBO J3, 2723-2730.
Voinnet O, Rivas S, Baulcombe DC. (2000) An enhanced transient expression system in plants based on suppression of gene silencing by the p19 protein of tomato bushy stunt virus. Plant J 33, 949-956.
Wassenegger, M., Heimes, S., Riedel, L., and Sanger, H. L. (1994). RNA-directed de novo methylation of genomic sequences in plants. Cell 76, 567-576.
Willmann, M. R., Endres, M. W., Cook, R. T., and Gregory, B. D. (2011). The Functions of RNA-Dependent RNA Polymerases in Arabidopsis. The Arabidopsis Book 9, e0146.
Wu, H. W., Lin, S. S., Chen, K. C., Yeh, S. D., and Chua, N. H. (2010). Discriminating mutations of HC-Pro of Zucchini yellow mosaic virus with differential effects on small RNA pathways involved in viral pathogenicity and symptom development. Molecular Plant-Microbe Interactions 23, 17-28.
Yang M., and Sank F. D. (1995). The too many mouths and four lips Mutations Affect Stomatal Production in Arabidopsis. The Plant Cell 7, 2227-2239
Zhang, X., Henriques, R., Lin, S. S., Niu, Q. W., and Chua, N. H. (2006). Agrobacterium-mediated transformation of Arabidopsis thaliana using the floral dip method. Nature Protocols 1, 641-646.
Zilberman, D., Gehring, M., Tran, R. K., Ballinger, T., and Henikoff, S. (2006). Genome-wide analysis of Arabidopsis thaliana DNA methylation uncovers an interdependence between methylation and transcription. Nature Genetics 39, 61-69.

1. An isolated silencing enhancer comprising the nucleotide sequence set forth in SEQ ID NO:6.

2. The isolated silencing enhancer of claim 1, wherein the silencing enhancer further comprises at the 3′ end from 1-1342 of the contiguous nucleotides set forth in SEQ ID NO:1.

3. The isolated silencing enhancer of claim 1, wherein the silencing enhancer comprises the nucleotide sequence set forth in SEQ ID NO:5.

4. The isolated silencing enhancer of claim 1, wherein the silencing enhancer comprises the nucleotide sequence set forth in SEQ ID NO:4.

5. The isolated silencing enhancer of claim 1, wherein the silencing enhancer comprises the nucleotide sequence set forth in SEQ ID NO:3.

6. The isolated silencing enhancer of claim 1, wherein the silencing enhancer comprises the nucleotide sequence set forth in SEQ ID NO:18.

7. A nucleic acid construct comprising a plant operable promoter operatively linked to a nucleic acid molecule that comprises the silencing enhancer of claim 1.

8. The nucleic acid construct of claim 7, wherein the plant operable promoter is a heterologous plant operable promoter.

9. The nucleic acid construct of claim 7, further comprising a nucleic acid silencer molecule operatively linked to a nucleic acid molecule comprising the silencing enhancer.

10. A transgenic plant cell comprising the nucleic acid construct of claim 9 stably integrated in its genome.

11. A transgenic plant comprising the nucleic acid construct of claim 9 stably integrated in its genome.

12. A method of preparing a transgenic plant having more effective gene silencing of a gene of interest comprising introducing the nucleic acid construct of claim 9 into a plant, wherein the transgenic plant has the nucleic acid construct stably integrated in its genome.

13. A method of preparing a transgenic plant having more effective gene silencing of a gene of interest comprising transfecting the nucleic acid construct of claim 9 into a plant cell or plant cells and regenerating a transgenic plant from the transfected plant cell or transfected plant cells, wherein the transgenic plant has the nucleic acid construct stably integrated in its genome.

14. A method of enhancing the reduction of gene expression of a plant gene in a plant cell comprising culturing the transgenic plant cell of claim 10 under conditions suitable for expression of the nucleic acid silencer molecule, whereby reduced expression of the plant gene is enhanced with respect to a corresponding transgenic plant cell not comprising the silencing enhancer.

15. A method of enhancing the reduction of gene expression of a plant gene in a plant comprising growing the transgenic plant of claim 11 under conditions suitable for expression of the nucleic acid silencer molecule, whereby reduced expression of the plant gene is enhanced with respect to a corresponding transgenic plant not comprising the silencing enhancer.

16. A method of enhancing the reduction of gene expression of a plant gene in a plant cell comprising:
transforming a plant cell with the nucleic acid construct of claim 9 to produce a transgenic plant cell having the nucleic acid construct stably integrated in its genome; and
culturing the transgenic plant cell under conditions suitable for expression of the nucleic acid silencer molecule, whereby reduced expression of the plant gene is enhanced with respect to a corresponding transgenic plant cell not comprising the silencing enhancer.

17. A method of enhancing the reduction of gene expression of a plant gene in a plant comprising:
transforming a plant cell with the nucleic acid construct of claim 9 to produce a transgenic plant cell having the nucleic acid construct stably integrated in its genome;
regenerating a transgenic plant from the transgenic plant cell, wherein the transgenic plant has the nucleic acid construct stably integrated in its genome; and
growing the transgenic plant under conditions suitable for expression the nucleic acid silencer molecule, whereby reduced expression of the plant gene is enhanced with respect to a corresponding transgenic plant not comprising the silencing enhancer.

18. A method to identify a putative silencing enhancer useful for more effectively silencing a gene of interest in a plant, the method comprising:
(a) transforming a first plant cell with a first nucleic acid construct comprising a plant operable promoter operatively linked to a nucleic acid comprising a putative silencing enhancer operatively linked to a nucleic acid silencer molecule;
(b) transforming a second plant cell with a second nucleic acid construct comprising a plant operable promoter operatively linked to a nucleic acid silencer molecule;
(c) determining the level of gene silencing in the first transformed plant cell and in the second transformed plant cell; and
(d) identifying the putative silencing enhancer as a silencing enhancer if the level of gene silencing is greater in the first transformed plant cell than in the second transformed plant cell.

19. The method of claim 18, which further comprises:
(a1) culturing the first transformed plant cells under conditions suitable for expression of the putative silencing enhancer and the nucleic acid silencer molecule; and
(b1) culturing the second transformed plant cells under conditions suitable for expression of the nucleic acid silencer molecule.

20. The method of claim 18, which further comprises:
(a1) regenerating a first transformed plant from the first transformed plant cell, wherein the first transformed plant comprises the first nucleic acid construct;
(a2) growing the first transformed plant under conditions suitable for expression of the nucleic acid silencer molecule in cells of the first transformed plant;
(b1) regenerating a second transformed plant from the second transformed plant cell, wherein the second transformed plant comprises the second nucleic acid construct; and
(b2) growing the second transformed plant under conditions suitable for expression of the nucleic acid silencer molecule in cells of the second transformed plant.


SEQUENCE LISTING (44 sequences)

SEQ ID NO: 1 - DNA, 1731 bp, Arabidopsis thaliana
gaaaatgaggaaagtgttgtgccaaatcacgaattagtacccatgtctgttgatgctctaacaattctttccacaagaccacaactccacaagtaacccgcgctatgttttcacgggtatcgacaatgttctcaggttccactataaactcattatccattgagaatttagtgcgcagaggagttgcttcaagttgattgaccaatacttttttcaattgttagacataaacactgaatacaaatctttgaacaacctgacttagatcatcctcattgcacaataaaacacagttttttttttttttttttttaaatgaaaaataaaataaaatgttatttagtgcttttaggaggaagactcactgtctttcatgtctctgcaaagacattttcttacttttaaagacatcacacttgactaataaatgactcaaattaaaataatgttaatttttataataaactataataaagacatctctaaaaaagtcaccaatgagaatgctcttaggaaccaaattttgcatgaggaaaactgttagctaagttagggtctggtttagaactcaagtccaactttcaaaatttaaaagaactaacttagtcaggttacgggttcttgggaatacgtttgtagtcgaaagaactgttttctcgatcgaaatttttggtaaaacatgaatgaaagaaaggtgatctccgacgaaaaattaataacctaatttatcatgaggttttgtagactaattttctttttactaaagtagtgtatttaagactcggtaatcaattaactatgttcagaatttttggttgtttggattgacttttgggtaaaagaatttagaattgcaaagaattgcaaacaatcggtttgttcggattaggtaaaagcattccgaattgaaaacctaagtttgtcttggacccctaaaatttaagcttaatttatcaatttcggtacatttgcaaagttcagtccaattcaaatattcggtttagaccaaaagttcgaataatgaaaaaaaaacatctgaaagttgtggaatttttttttaaagcaaattggtaaaccataagaacttctttaaaagtccaccacatcatttatccttcaaaatctccccaaatacgaaatcaagcaagtcttccaaaatttagaatctccaatacaccatgttgggaaacaaaccaactttttacatccgaagattatagatatattgtctaatatatcaatcaagtccaactccaattagaagaaagtttttttggaaaaaagccctaaacaaatttgtgcaagttttaccacttggacccaaagcggacaatatcatcctaatcgaaaaagttggaatgggcttggagagcccaacaaccctattttttagaaaacaataaatttatttgcaaaacgacatgaaaatcatcgtacaaaacattcaagatatatgatgaattatttgattattaccacgtcagtagttatgaagataagattttacagtacacgttgatataaagatgagatttcgctgtacacgtcagttatttagataaaatttcacagtacacgtcagttataaagataagatttcacagtacacatcagttataaagataagatttcacagtacacgtcagttataaagataagatttcattgtacacgttagttataaagataagatttcacaatacacgtcagccctaacacaaaacatactag

SEQ ID NO: 2 - DNA, 475 bp, Arabidopsis thaliana
tgttgctccatgggcatgtgcttttcgtatgcacagaccacgcgtccgtttattcagattctcgtgttacaacaaaataatgattttaaggttcagaatcaaacatatgaaactttttatcaattgctatctacggtttgttacaaaaacaaatacagtgcccagttcaaaatacaaattgaataatagcccaacatatcgtaaatgggaaaatgggctcagcccaaataaaaaaaaagtaaacgataaggagacgtcgggtcccgttcacgaagcggtcggatctagggcccaacacaagacgaatgaagggaatatctcgtgaaagcaataaatgcagaaatcgagaatcatgaattttatttatttcgctcctcgagttatcccgtgaaagtcataaagacattgcgaacgtttttatggacccaccaattgtttcttcagaattatacatatacacactgacaatttcgaa

SEQ ID NO: 3 - DNA, 1466 bp, Arabidopsis thaliana
gaaaatgaggaaagtgttgtgccaaatcacgaattagtacccatgtctgttgatgctctaacaattctttccacaagaccacaactccacaagtaacccgcgctatgttttcacgggtatcgacaatgttctcaggttccactataaactcattatccattgagaatttagtgcgcagaggagttgcttcaagttgattgaccaatacttttttcaattgttagacataaacactgaatacaaatctttgaacaacctgacttagatcatcctcattgcacaataaaacacagttttttttttttttttttttaaatgaaaaataaaataaaatgttatttagtgcttttaggaggaagactcactgtctttcatgtctctgcaaagacattttcttacttttaaagacatcacacttgactaataaatgactcaaattaaaataatgttaatttttataataaactataataaagacatctctaaaaaagtcaccaatgagaatgctcttaggaaccaaattttgcatgaggaaaactgttagctaagttagggtctggtttagaactcaagtccaactttcaaaatttaaaagaactaacttagtcaggttacgggttcttgggaatacgtttgtagtcgaaagaactgttttctcgatcgaaatttttggtaaaacatgaatgaaagaaaggtgatctccgacgaaaaattaataacctaatttatcatgaggttttgtagactaattttctttttactaaagtagtgtatttaagactcggtaatcaattaactatgttcagaatttttggttgtttggattgacttttgggtaaaagaatttagaattgcaaagaattgcaaacaatcggtttgttcggattaggtaaaagcattccgaattgaaaacctaagtttgtcttggacccctaaaatttaagcttaatttatcaatttcggtacatttgcaaagttcagtccaattcaaatattcggtttagaccaaaagttcgaataatgaaaaaaaaacatctgaaagttgtggaatttttttttaaagcaaattggtaaaccataagaacttctttaaaagtccaccacatcatttatccttcaaaatctccccaaatacgaaatcaagcaagtcttccgaaatttagaatctccaatacaccatgttgggaaacaaaccaactttttacatccgaagattatagatatattgtctaatatatcaatcaagtccaactccaattagaagaaagtttttttggaaaaaagccctaaacaaatttgtgcaagttttaccacttggacccaaagcggacaatatcatcctaatcgaaaaagttggaatgggcttggagagcccaacaaccctattttttagaaaacaataaatttatttgcaaaacgacatgaaaatcatcgtacaaaacattcaagatatatgatgaattatt

SEQ ID NO: 4 - DNA, 1180 bp, Arabidopsis thaliana
gaaaatgaggaaagtgttgtgccaaatcacgaattagtacccatgtctgttgatgctctaacaattctttccacaagaccacaactccacaagtaacccgcgctatgttttcacgggtatcgacaatgttctcaggttccactataaactcattatccattgagaatttagtgcgcagaggagttgcttcaagttgattgaccaatacttttttcaattgttagacataaacactgaatacaaatctttgaacaacctgacttagatcatcctcattgcacaataaaacacagttttttttttttttttttttaaatgaaaaataaaataaaatgttatttagtgcttttaggaggaagactcactgtctttcatgtctctgcaaagacattttcttacttttaaagacatcacacttgactaataaatgactcaaattaaaataatgttaatttttataataaactataataaagacatctctaaaaaagtcaccaatgagaatgctcttaggaaccaaattttgcatgaggaaaactgttagctaagttagggtctggtttagaactcaagtccaactttcaaaatttaaaagaactaacttagtcaggttacgggttcttgggaatacgtttgtagtcgaaagaactgttttctcgatcgaaatttttggtaaaacatgaatgaaagaaaggtgatctccgacgaaaaattaataacctaatttatcatgaggttttgtagactaattttctttttactaaagtagtgtatttaagactcggtaatcaattaactatgttcagaatttttggttgtttggattgacttttgggtaaaagaatttagaattgcaaagaattgcaaacaatcggtttgttcggattaggtaaaagcattccgaattgaaaacctaagtttgtcttggacccctaaaatttaagcttaatttatcaatttcggtacatttgcaaagttcagtccaattcaaatattcggtttagaccaaaagttcgaataatgaaaaaaaaacatctgaaagttgtggaatttttttttaaagcaaattggtaaaccataagaacttctttaaaagtccaccacatcatttatccttcaaaatctccccaaatacgaaatcaagcaagtcttccaaaatttagaatctccaatacaccat

SEQ ID NO: 5 - DNA, 709 bp, Arabidopsis thaliana
gaaaatgaggaaagtgttgtgccaaatcacgaattagtacccatgtctgttgatgctctaacaattctttccacaagaccacaactccacaagtaacccgcgctatgttttcacgggtatcgacaatgttctcaggttccactataaactcattatccattgagaatttagtgcgcagaggagttgcttcaagttgattgaccaatacttttttcaattgttagacataaacactgaatacaaatctttgaacaacctgacttagatcatcctcattgcacaataaaacacagttttttttttttttttttttaaatgaaaaataaaataaaatgttatttagtgcttttaggaggaagactcactgtctttcatgtctctgcaaagacattttcttacttttaaagacatcacacttgactaataaatgactcaaattaaaataatgttaatttttataataaactataataaagacatctctaaaaaagtcaccaatgagaatgctcttaggaaccaaattttgcatgaggaaaactgttagctaagttagggtctggtttagaactcaagtccaactttcaaaatttaaaagaactaacttagtcaggttacgggttcttgggaatacgtttgtagtcgaaagaactgttttctcgatcgaaatttttggtaaaacatgaatgaaagaaaggtgatctccgacgaa

SEQ ID NO: 6 - DNA, 389 bp, Arabidopsis thaliana
gaaaatgaggaaagtgttgtgccaaatcacgaattagtacccatgtctgttgatgctctaacaattctttccacaagaccacaactccacaagtaacccgcgctatgttttcacgggtatcgacaatgttctcaggttccactataaactcattatccattgagaatttagtgcgcagaggagttgcttcaagttgattgaccaatacttttttcaattgttagacataaacactgaatacaaatctttgaacaacctgacttagatcatcctcattgcacaataaaacacagttttttttttttttttttttaaatgaaaaataaaataaaatgttatttagtgcttttaggaggaagactcactgtctttcatgtctctgcaaagac

SEQ ID NO: 7 - DNA, 1342 bp, Arabidopsis thaliana
attttcttacttttaaagacatcacacttgactaataaatgactcaaattaaaataatgttaatttttataataaactataataaagacatctctaaaaaagtcaccaatgagaatgctcttaggaaccaaattttgcatgaggaaaactgttagctaagttagggtctggtttagaactcaagtccaactttcaaaatttaaaagaactaacttagtcaggttacgggttcttgggaatacgtttgtagtcgaaagaactgttttctcgatcgaaatttttggtaaaacatgaatgaaagaaaggtgatctccgacgaaaaattaataacctaatttatcatgaggttttgtagactaattttctttttactaaagtagtgtatttaagactcggtaatcaattaactatgttcagaatttttggttgtttggattgacttttgggtaaaagaatttagaattgcaaagaattgcaaacaatcggtttgttcggattaggtaaaagcattccgaattgaaaacctaagtttgtcttggacccctaaaatttaagcttaatttatcaatttcggtacatttgcaaagttcagtccaattcaaatattcggtttagaccaaaagttcgaataatgaaaaaaaaacatctgaaagttgtggaatttttttttaaagcaaattggtaaaccataagaacttctttaaaagtccaccacatcatttatccttcaaaatctccccaaatacgaaatcaagcaagtcttccaaaatttagaatctccaatacaccatgttgggaaacaaaccaactttttacatccgaagattatagatatattgtctaatatatcaatcaagtccaactccaattagaagaaagtttttttggaaaaaagccctaaacaaatttgtgcaagttttaccacttggacccaaagcggacaatatcatcctaatcgaaaaagttggaatgggcttggagagcccaacaaccctattttttagaaaacaataaatttatttgcaaaacgacatgaaaatcatcgtacaaaacattcaagatatatgatgaattatttgattattaccacgtcagtagttatgaagataagattttacagtacacgttgatataaagatgagatttcgctgtacacgtcagttatttagataaaatttcacagtacacgtcagttataaagataagatttcacagtacacatcagttataaagataagatttcacagtacacgtcagttataaagataagatttcattgtacacgttagttataaagataagatttcacaatacacgtcagccctaacacaaaacatactag

SEQ ID NO: 8 - DNA, 1022 bp, Arabidopsis thaliana
aaattaataacctaatttatcatgaggttttgtagactaattttctttttactaaagtagtgtatttaagactcggtaatcaattaactatgttcagaatttttggttgtttggattgacttttgggtaaaagaatttagaattgcaaagaattgcaaacaatcggtttgttcggattaggtaaaagcattccgaattgaaaacctaagtttgtcttggacccctaaaatttaagcttaatttatcaatttcggtacatttgcaaagttcagtccaattcaaatattcggtttagaccaaaagttcgaataatgaaaaaaaaacatctgaaagttgtggaatttttttttaaagcaaattggtaaaccataagaacttctttaaaagtccaccacatcatttatccttcaaaatctccccaaatacgaaatcaagcaagtcttccaaaatttagaatctccaatacaccatgttgggaaacaaaccaactttttacatccgaagattatagatatattgtctaatatatcaatcaagtccaactccaattagaagaaagtttttttggaaaaaagccctaaacaaatttgtgcaagttttaccacttggacccaaagcggacaatatcatcctaatcgaaaaagttggaatgggcttggagagcccaacaaccctattttttagaaaacaataaatttatttgcaaaacgacatgaaaatcatcgtacaaaacattcaagatatatgatgaattatttgattattaccacgtcagtagttatgaagataagattttacagtacacgttgatataaagatgagatttcgctgtacacgtcagttatttagataaaatttcacagtacacgtcagttataaagataagatttcacagtacacatcagttataaagataagatttcacagtacacgtcagttataaagataagatttcattgtacacgttagttataaagataagatttcacaatacacgtcagccctaacacaaaacatactag

SEQ ID NO: 9 - DNA, 551 bp, Arabidopsis thaliana
gttgggaaacaaaccaactttttacatccgaagattatagatatattgtctaatatatcaatcaagtccaactccaattagaagaaagtttttttggaaaaaagccctaaacaaatttgtgcaagttttaccacttggacccaaagcggacaatatcatcctaatcgaaaaagttggaatgggcttggagagcccaacaaccctattttttagaaaacaataaatttatttgcaaaacgacatgaaaatcatcgtacaaaacattcaagatatatgatgaattatttgattattaccacgtcagtagttatgaagataagattttacagtacacgttgatataaagatgagatttcgctgtacacgtcagttatttagataaaatttcacagtacacgtcagttataaagataagatttcacagtacacatcagttataaagataagatttcacagtacacgtcagttataaagataagatttcattgtacacgttagttataaagataagatttcacaatacacgtcagccctaacacaaaacatactag

SEQ ID NO: 10 - DNA, 265 bp, Arabidopsis thaliana
tgattattaccacgtcagtagttatgaagataagattttacagtacacgttgatataaagatgagatttcgctgtacacgtcagttatttagataaaatttcacagtacacgtcagttataaagataagatttcacagtacacatcagttataaagataagatttcacagtacacgtcagttataaagataagatttcattgtacacgttagttataaagataagatttcacaatacacgtcagccctaacacaaaacatactag

SEQ ID NO: 11 - DNA, 475 bp, Arabidopsis thaliana
ttcgaaattgtcagtgtgtatatgtataattctgaagaaacaattggtgggtccataaaaacgttcgcaatgtctttatgactttcacgggataactcgaggagcgaaataaataaaattcatgattctcgatttctgcatttattgctttcacgagatattcccttcattcgtcttgtgttgggccctagatccgaccgcttcgtgaacgggacccgacgtctccttatcgtttactttttttttatttgggctgagcccattttcccatttacgatatgttgggctattattcaatttgtattttgaactgggcactgtatttgtttttgtaacaaaccgtagatagcaattgataaaaagtttcatatgtttgattctgaaccttaaaatcattattttgttgtaacacgagaatctgaataaacggacgcgtggtctgtgcatacgaaaagcacatgcccatggagcaaca

SEQ ID NO: 12 - DNA, 1144 bp, Arabidopsis thaliana
ctgttgctccatgggcatgtgcttttcgtatgcacagaccacgcgtccgtttattcagattctcgtgttacaacaaaataatgattttaaggttcagaatcaaacatatgaaactttttatcaattgctatctacggtttgttacaaaaacaaatacagtgcccagttcaaaatacaaattgaataatagcccaacatatcgtaaatgggaaaatgggctcagcccaaataaaaaaaaagtaaacgataaggagacgtcgggtcccgttcacgaagcggtcggatctagggcccaacacaagacgaatgaagggaatatctcgtgaaagcaataaatgcagaaatcgagaatcatgaattttatttatttcgctcctcgagttatcccgtgaaagtcataaagacattgcgaacgtttttatggacccaccaattgtttcttcagaattatacatatacacactgacaatttcgaaactagggccatgcacaacaacacgtactcacaaaggtgtcaatcgagcagcccaaaacattcaccaactcaacccatcatgagcccacacatttgttgtttctaacccaacctcaaacccgtattctcttccgccacctcatttttgtttagttcaacacccgtcaaactgcatcccaccccgtggactaggttcgaaattgtcagtgtgtatatgtataattctgaagaaacaattggtgggtccataaaaacgttcgcaatgtctttatgactttcacgggataactcgaggagcgaaataaataaaattcatgattctcgatttctgcatttattgctttcacgagatattcccttcattcgtcttgtgttgggccctagatccgaccgcttcgtgaacgggacccgacgtctccttatcgtttactttttttttatttgggctgagcccattttcccatttacgatatgttgggctattattcaatttgtattttgaactgggcactgtatttgtttttgtaacaaaccgtagatagcaattgataaaaagtttcatatgtttgattctgaaccttaaaatcattattttgttgtaacacgagaatctgaataaacggacgcgtggtctgtgcatacgaaaagcacatgcccatggagcaacag

SEQ ID NO: 13 - DNA, 1038 bp, Arabidopsis thaliana
atcagttaacacttattaagaacaaaaatgtggtttcttgtgagaaaaatggtttaataaaaatccgtgattgatagaagaaaaagatcaaaataaatggttggtgacgggtgatcttaaaaatgttgaaattaaggtgtgtcgtcgttatacgcggtaaatagatagatagaaaaatagaagtccaatgcaagagacttaacttaatcatcccaattaattgattgcattaacttgtacttgtattttccgtccgccacctaatttgattaataatataataaagattacaattgaaaacataaacaagagaaaatccgcacgaatctaccaaagtgcatcacgtttgggtatccatacacgtgaccaccagtccaccacaacacaatgtctgtagatattttaatgtttcacatgatagaagaagccaaacgtaagaactctcttttccacttttagccctttccccgcctaccactgcttacgacttgtgtaagtggcaaactagtaataatagagacgaaacttaaatataaaaaagttgaatccaaccaagttggtgttaatcaaatggttaagttataatggtgaaagatttgccatgtgtattgtattaagagttaagaccaaggtttggttcccatcacttacgattctttcttttcatatgattctaaagttagttattataaacatcttaatttactacacaatattcggtaatttctacatattttagagattagtttgagtttcaatccatactttactagtgattataaattaatatacgtacttttcgactataaagtgaaactaagtaaattagaacgtgatattaaaaagttaatgttcactgttatatttttttcacaagtaaaaaatgggttatttgcggtaaataaaaataccagatattttgaattgattaaaaaggttgaaataagagaggaggggaaagaaaagaaggtgggggcccagtatgaaagggaaaggtgtcatcaaatcatctctctctctctctctctaccttcgacc

SEQ ID NO: 14 - DNA, 476 bp, Arabidopsis thaliana
tcgagactaaatttcagggacaacgagcagcacgaaactaagtttaggagaaagtgcatcttagcattgatcgaacatttcttactcaaatctacgcataaacgtcacctctaacacagaaatgttcatcgattatgatcaaccgatcgtcaatcgtcgaaccttagcaaaccgaagctaaaacaacgcctgacagtgagattctactcaatcgacgagcaacgagggtaaattcttaccgattgaatcgctttgcagttgtatatgtagtaggcgctaacgataaacgttcaacggcaacaagacgacgacaccggagagaaaatcgccgacggaactcgaaggggggagattttgaaattagctgggctccgaatgatttgacttgggcctttatattcttatgtttggtatatatatatagaagctcgcctttttttttggtacggctattttattttctatatcatttttga

SEQ ID NO: 15 - DNA, 263 bp, Arabidopsis thaliana
caaaagccatgcacaacaacacgtactcacaaaggcgtcaatcgagcagcccaaaacattcaccaactcaacccatcatgagcccacacatttgttgtttctaacccaacctcaaactcgtattctcttccgccacctcatttttgtttatttcaacacccgtcaaactgcatcccaccccgtggccaaatgttcatgcatgttaacaagacctatgactataaatatctgcaatctcggcccaagttttcatcatcaagaac

SEQ ID NO: 16 - DNA, 720 bp, Cauliflower mosaic virus
cgccctgagtgcttgcggcagcgtgaagcttgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaa

SEQ ID NO: 17 - DNA, 395 bp, Tobacco rattle virus
atgtcccgaagacattaaactacggttctttaagtagatccgtgtctgaagttttaggttcaatttaaacctacgagattgacattctcgactgatcttgattgatcggtaagtcttttgtaatttaattttctttttgattttattttaaattgttatctgtttctgtgtatagactgtttgagatcggcgtttggccgactcattgtcttaccataggggaacggactttgtttgtgttgttattttatttgtattttattaaaattctcaacgatctgaaaaagcctcgcggctaagagattgttggggggtgagtaagtacttttaaagtgatgatggttacaaaggcaaaaggggtaaaacccctcgcctacgtaagcgttattacgccc

SEQ ID NO: 18 - DNA, 1487 bp, Arabidopsis thaliana
gaaaatgaggaaagtgttgtgccaaatcacgaattagtacccatgtctgttgatgctctaacaattctttccacaagaccacaactccacaagtaacccgcgctatgttttcacgggtatcgacaatgttctcaggttccactataaactcattatccattgagaatttagtgcgcagaggagttgcttcaagttgattgaccaatacttttttcaattgttagacataaacactgaatacaaatctttgaacaacctgacttagatcatcctcattgcacaataaaacacagttttttttttttttttttttaaatgaaaaataaaataaaatgttatttagtgcttttaggaggaagactcactgtctttcatgtctctgcaaagacattttcttacttttaaagacatcacacttgactaataaatgactcaaattaaaataatgttaatttttataataaactataataaagacatctctaaaaaagtcaccaatgagaatgctcttaggaaccaaattttgcatgaggaaaactgttagctaagttagggtctggtttagaactcaagtccaactttcaaaatttaaaagaactaacttagtcaggttacgggttcttgggaatacgtttgtagtcgaaagaactgttttctcgatcgaaatttttggtaaaacatgaatgaaagaaaggtgatctccgacgaaaaattaataacctaatttatcatgaggttttgtagactaattttctttttactaaagtagtgtatttaagactcggtaatcaattaactatgttcagaatttttggttgtttggattgacttttgggtaaaagaatttagaattgcaaagaattgcaaacaatcggtttgttcggattaggtaaaagcattccgaattgaaaacctaagtttgtcttggacccctaaaatttaagcttaatttatcaatttcggtacatttgcaaagttcagtccaattcaaatattcggtttagaccaaaagttcgaataatgaaaaaaaaacatctgaaagttgtggaatttttttttaaagcaaattggtaaaccataagaacttctttaaaagtccaccacatcatttatccttcaaaatctccccaaatacgaaatcaagcaagtcttccgaaatttagaatctccaatacaccatgttgggaaacaaaccaactttttacatccgaagattatagatatattgtctaatatatcaatcaagtccaactccaattagaagaaagtttttttggaaaaaagccctaaacaaatttgtgcaagttttaccacttggacccaaagcggacaatatcatcctaatcgaaaaagttggaatgggcttggagagcccaacaaccctattttttagaaaacaataaatttatttgcaaaacgacatgaaaatcatcgtacaaaacattcaagatatatgatgaattatttgattattaccacgtcagtag

SEQ ID NO: 19 - DNA, 31 bp, Artificial Sequence
  oligonucleotide primer
cacctaggaaaatgaggaaagtgttgtgcca

SEQ ID NO: 20 - DNA, 59 bp, Artificial Sequence
  oligonucleotide primer
cggcgcgcccacccttaatagtcgactctagactagtctagtatgttttgtgttagggc

SEQ ID NO: 21 - DNA, 31 bp, Artificial Sequence; identical to SEQ ID NO: 19
  oligonucleotide primer

SEQ ID NO: 22 - DNA, 41 bp, Artificial Sequence
  oligonucleotide primer
tactcgagtctagactagtaataattcatcatatatcttga

SEQ ID NO: 23 - DNA, 31 bp, Artificial Sequence; identical to SEQ ID NO: 19
  oligonucleotide primer

SEQ ID NO: 24 - DNA, 38 bp, Artificial Sequence
  oligonucleotide primer
actcgagtctagactagtatggtgtattggagattcta

SEQ ID NO: 25 - DNA, 31 bp, Artificial Sequence; identical to SEQ ID NO: 19
  oligonucleotide primer

SEQ ID NO: 26 - DNA, 40 bp, Artificial Sequence
  oligonucleotide primer
tactcgagtctagactagtttcgtcggagatcacctttct

SEQ ID NO: 27 - DNA, 31 bp, Artificial Sequence; identical to SEQ ID NO: 19
  oligonucleotide primer

SEQ ID NO: 28 - DNA, 41 bp, Artificial Sequence
  oligonucleotide primer
tactcgagtctagactagtgtctttgcagagacatgaaaga

SEQ ID NO: 29 - DNA, 30 bp, Artificial Sequence
  oligonucleotide primer
cacctaggaattttcttacttttaaagaca

SEQ ID NO: 30 - DNA, 59 bp, Artificial Sequence; identical to SEQ ID NO: 20
  oligonucleotide primer

SEQ ID NO: 31 - DNA, 30 bp, Artificial Sequence
  oligonucleotide primer
cacctaggaaattaataacctaatttatca

SEQ ID NO: 32 - DNA, 59 bp, Artificial Sequence; identical to SEQ ID NO: 20
  oligonucleotide primer

SEQ ID NO: 33 - DNA, 34 bp, Artificial Sequence
  oligonucleotide primer
cacctagggttgggaaacaaaccaactttttaca

SEQ ID NO: 34 - DNA, 59 bp, Artificial Sequence; identical to SEQ ID NO: 20
  oligonucleotide primer

SEQ ID NO: 35 - DNA, 28 bp, Artificial Sequence
  oligonucleotide primer
cacctaggtgattattaccacgtcagta

SEQ ID NO: 36 - DNA, 59 bp, Artificial Sequence; identical to SEQ ID NO: 20
  oligonucleotide primer

SEQ ID NO: 37 - DNA, 31 bp, Artificial Sequence
  oligonucleotide primer
cacctaggctgttgctccatgggcatgtgct

SEQ ID NO: 38 - DNA, 57 bp, Artificial Sequence
  oligonucleotide primer
cggcgcgcccacccttaatagtcgactctagactagtttcgaaattgtcagtgtgta

SEQ ID NO: 39 - DNA, 28 bp, Artificial Sequence
  oligonucleotide primer
cacctaggttcgaaattgtcagtgtgta

SEQ ID NO: 40 - DNA, 59 bp, Artificial Sequence
  oligonucleotide primer
cggcgcgcccacccttaatagtcgactctagactagtctgttgctccatgggcatgtgc

SEQ ID NO: 41 - DNA, 29 bp, Artificial Sequence
  oligonucleotide primer
cacctaggatcagttaacacttattaaga

SEQ ID NO: 42 - DNA, 53 bp, Artificial Sequence
  oligonucleotide primer
tcggcgcgcccacccttagtcgactctagaggtcgaaggtagagagagagaga

SEQ ID NO: 43 - DNA, 28 bp, Artificial Sequence
  oligonucleotide primer
cacctaggtcgagactaaatttcaggga

SEQ ID NO: 44 - DNA, 49 bp, Artificial Sequence
  oligonucleotide primer
actcgagtcgactctagactagttcaaaaatgatatagaaaataaaata